(19) United States
(12) Patent Application Publication
(10) Pub. No.: US 2009/0061423 A1
(43) Pub. Date: Mar. 5, 2009

(54) PHARMACOGENOMIC MARKERS FOR PROGNOSIS OF SOLID TUMORS

(75) Inventors: Michael Edward Burczynski, Collegeville, PA (US); Frederick William Immermann, Suffern, NY (US); Andrew Louis Strahs, Maynard, MA (US); Natalie Constance Twine, Goffstown, NH (US); Donna Karen Slonim, North Andover, MA (US); William Liapord Trepicchio, Andover, MA (US); Andrew Joseph Dorner, Lexington, MA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 11/816,214

(22) PCT Filed: Feb. 17, 2006
(86) PCT No.: PCT/US2006/005772

§ 371 Date: Jun. 5, 2008

Related U.S. Application Data
(60) Provisional application No. 60/654,082, filed on Feb. 18, 2005.

(51) Int. Cl. C12Q 1/68

(52) U.S. Cl. 435/6

(57) ABSTRACT

The present invention provides methods, systems and equipment for prognosis or evaluation of treatment of solid tumors. Gene markers that are prognostic of solid tumors can be identified according to the present invention. Each gene marker has altered expression patterns in PBMCs of solid tumor patients following initiation of an anti-cancer treatment, and the magnitudes of these alterations are correlated with clinical outcomes of these patients. In one embodiment, a Cox proportional hazards model is used to determine the correlations between clinical outcomes of RCC patients and gene expression changes in PBMCs of these patients during the course of a CCI-779 treatment. Non-limiting examples of genes identified by the Cox model are depicted in Tables 4A3 4B, 5 A and 5B. These genes can be used as surrogate markers for prognosis of RCC. They can also be used as pharmacogenomic indicators for the efficacy of CCI-779 or other anti-cancer drugs.

### CROSS-REFERENCE TO RELATED APPLICATIONS

This application claims the benefit of U.S. Ser. No. 60/654,082, filed Feb. 18, 2005.

### TECHNICAL FIELD

The present invention relates to gene markers and methods of using the same for prognosis of solid tumors.

### BACKGROUND

Expression profiling studies in primary tissues have demonstrated that there exist transcriptional differences between normal and malignant tissues. See, for example, Su, et al., CANCER RES., 61:7388-7393 (2001); and Ramaswamy, et al., PROC. NATL. ACAD. SCI. U.S.A., 98:15149-15151 (2001). Recent clinical analyses have also identified expression profiles from tumors that appear to be highly correlated with certain measures of clinical outcomes. One study has demonstrated that expression profiling of primary tumor biopsies yields prognostic “signatures” that rival or may even out-perform currently accepted standard measures of risk in cancer patients. See van de Vijver, et al., N ENGL J MED, 347:1999-2009 (2002).

Although transcriptional or other biochemical changes in the primary tumor tissue may represent the best opportunity to identify prognostic evidence, in many oncology scenarios the primary tumor is resected prior to initiation of chemotherapy. In these settings, it is therefore desirable to determine whether responses in some other “surrogate” tissues can provide indications of patient outcome.

### SUMMARY OF THE INVENTION

The present invention features gene markers in peripheral blood mononuclear cells (PBMCs) that can provide clues to eventual clinical outcome of solid tumor patients. Each gene marker has an altered expression pattern in PBMCs of solid tumor patients following initiation of an anti-cancer treatment, and the magnitude of this alteration is statistically significantly correlated with clinical outcome of the solid tumor patients. In many embodiments, the correlation between gene expression changes in PBMCs and patient outcomes is determined by a Cox proportional hazard model, a Spearman correlation, or a class-based correlation metric. The gene markers of the present invention can be used as surrogate markers for the prognosis of solid tumors. They can also be used as pharmacogenomic indicators for the efficacy of anti-cancer drugs.

In one aspect, the present invention provides methods for prognosis, or evaluation of the effectiveness of a treatment, of a solid tumor in a patient of interest. The methods comprise detecting a change in the expression level of at least one gene in peripheral blood cells of the patient of interest during the course of an anti-cancer treatment and comparing the detected change to a reference change. The expression level changes of the gene(s) in PBMCs of patients who have the same solid tumor and receive the same treatment as the patient of interest are correlated with clinical outcomes of these patients. Therefore, the magnitude of the expression level change in the patient of interest is indicative of the prognosis or effectiveness of the treatment of that patient. In many embodiments, the reference change has an empirically or experimentally determined value. The patient of interest is considered to have a good or poor prognosis if the expression level change in the patient of interest is greater or lesser than the reference change. In many other embodiments, the reference change is an expression level change of the gene(s) in peripheral blood cells of a reference patient who has the same solid tumor and receives the same treatment as the patient of interest. Other measures or criteria can also be used to calculate the reference change.

A variety of types of blood samples can be used to determine gene expression changes in a patient of interest. Examples of these blood samples include, but are not limited to, whole blood samples or samples comprising enriched or purified PBMCs. Other types of blood samples can also be used. Gene expression level changes in these samples are statistically significantly correlated with patient outcomes under an appropriate correlation model.

Solid tumors amenable to the present invention include, but are not limited to, renal cell carcinoma (RCC), prostate cancer, or head/neck cancer. Anti-cancer treatments that can be assessed according to the present invention include, but are not limited to, drug therapy, chemotherapy, hormone therapy, radiotherapy, immunotherapy, surgery, gene therapy, anti-angiogenesis therapy, palliative therapy, or other conventional or experimental therapies, or a combination thereof. Any time-associated clinical indictor can be used to evaluate the prognosis or effectiveness of a treatment of a patient of interest. Non-limitation examples of these clinical indictors include time to disease progression (TTP) or time to death (TTD).

A variety of correlation or statistical methods can be used to assess the correlations between peripheral blood gene expression changes during the course of an anti-cancer treatment and patient outcomes. These methods include, but are not limited to, the Cox proportional hazards model, the nearest-neighbor analysis, the significance analysis of microarrays (SAM) method, support vector machines, artificial neural networks, or other rank tests, survival analyses or correlation metrics.

In one embodiment, univariate Cox proportional hazards models are used to determine the correlations between gene expression level changes in PBMCs of RCC patients following initiation of a CCI-779 treatment and a temporal measurer of clinical outcomes of these patients (e.g., TTP or TTD). Non-limiting examples of prognostic genes identified by the Cox proportional hazards models are described in Tables 4A, 4B, 5A and 5B. These prognostic genes can be used for predicting clinical outcome, or evaluating the effectiveness of an anti-cancer treatment, of an RCC patient of interest.

In one embodiment, the estimated hazard ratio of a prognostic gene employed in the present invention is less than 1. As a consequence, a greater value of the change in the expression level of the gene in peripheral blood cells of a patient of interest is suggestive of a better prognosis of the patient. Conversely, a lesser value of the change in the patient of interest is indicative of a poorer prognosis.

In another embodiment, the hazard ratio of a prognostic gene employed in the present invention is greater than 1. As a result, a greater value of the change in the expression level of the gene in peripheral blood cells of a patient of interest is indicative of a poorer prognosis of the patient, and a lesser value of the change in the patient of interest is suggestive of a better prognosis.

The expression level change in a patient of interest can be measured from any reference point. The expression level change thus measured is statistically significantly correlated with patient outcome under an appropriate correlation model. In many instances, the expression level change of a prognostic gene is determined by measuring the alteration between the peripheral blood expression level of the gene at a specified time after initiation of an anti-cancer treatment and the baseline peripheral blood expression level of the gene. In one non-limiting example, the specified time is about 16 weeks after initiation of the treatment. A specified time of less than or greater than 16 weeks (e.g., 4, 8, 12, 20, 24, or 28 weeks after initiation of the treatment) can also be used.

The present invention also features use of two or more gene markers, or multivariate Cox models, for prognosis of solid tumors. In addition, the present invention features kits useful for prognosis of RCC or other solid tumors. Each kit includes or consists essentially of at least one probe for a prognostic gene of the present invention.

In another aspect, the present invention features methods of using logistic regression, ANOVA (analysis of variance), ANCOVA (analysis of covariance), MANOVA (multiple analysis of variance), or other correlation or statistical methods for prognosis, or evaluation of the effectiveness of a treatment, of a solid tumor in a patient of interest. These methods comprise detecting the expression level of at least one solid tumor prognostic gene in peripheral blood cells of the patient of interest at a specified time after initiation of an anti-cancer treatment and entering the expression level into a correlation or statistical model to determine the prognosis or effectiveness of the treatment of the patient of interest. The correlation or statistical model describes a statistically significant correlation between the expression levels of the solid tumor prognostic gene(s) in PBMCs of patients who have the same solid tumor and receive the same treatment as the patient of interest, and clinical outcomes of these patients. In many examples, the correlation or statistical model is capable of producing a qualitative prediction of the clinical outcome of the patient of interest (e.g., good or poor prognosis). Statistical models or analyses suitable for this purpose include, but are not limited to, logistic regression or class-based correlation metrics. In many other examples, the correlation or statistical model is capable of producing a quantitative prediction of the clinical outcome of the patient of interest (e.g., an estimated TTD or TTP). Statistical models or analyses suitable for this purpose include, but are not limited to, a variety of regression, ANOVA or ANCOVA models.

The expression levels used for prognosticating the patient of interest can be relative expression levels measured from baseline or another reference time point after initiation of the anti-cancer treatment. Absolute expression levels can also be used for prognosticating the patient of interest. In the latter case, expression levels at baseline or another specified reference time can be used as covariates in the prediction model.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### DETAILED DESCRIPTION

The present invention provides methods and systems for prognosis of RCC or other solid tumors. Solid tumor prognostic genes can be identified by the present invention. Each prognostic gene has altered expression profiles in PBMCs of solid tumor patients following initiation of an anti-cancer treatment, and the magnitudes of these alterations are correlated with clinical outcomes of these patients. In many embodiments, the expression profile alterations are measured from baseline, and the correlations between the expression profile alterations and patient outcomes are assessed by a Cox proportional hazards model.

The prognostic genes of the present invention can be used as surrogate markers for prognosis or monitoring the effectiveness of a treatment of a solid tumor patient of interest. Different patients may have distinct clinical responses to a treatment due to individual heterogeneity of the molecular mechanism of the disease. The identification of gene expression patterns that correlate with patient response allows clinicians to select treatments based on predicted patient response and thereby avoid adverse reactions. This provides improved safety of clinical trials and increased benefit/risk ratio for drugs and other anti-cancer treatments. Peripheral blood is a tissue that can be routinely obtained from patients in a minimally invasive manner. By determining the correlations between patient outcomes and gene expression changes in peripheral blood, the present invention represents a significant advance in clinical pharmacogenomics and solid tumor treatment.

Various aspects of the invention are described in further detail in the following subsections. The use of subsections is not meant to limit the invention. Each subsection may apply to any aspect of the invention. In this application, the singular forms “a” and “an” include plural reference unless the context clearly dictates otherwise, and the use of “or” means “and/or” unless stated otherwise.

### I. GENERAL METHODS FOR IDENTIFYING SOLID TUMOR PROGNOSTIC GENES

The present invention identifies statistically significant correlations between alterations in peripheral blood gene expression profiles and clinical outcomes of solid tumor patients. Genes with such correlations can be identified. These genes are solid tumor prognostic genes and can be used as surrogate markers for prognosis or evaluation of the effectiveness of a treatment of solid tumors.

Correlation analyses suitable for the present invention include, but are not limited to, the Cox proportional hazards model (Cox, JOURNAL OF THE ROYAL STATISTICAL SOCIETY, SERIES B 34:187 (1972)), the Speannan's rank correlation (Snedecor and Cochran, STATISTICAL METHODS (8^{th} edition, Iowa State University Press, Ames, Iowa, 503 pp, 1989)), the nearest-neighbor analysis (Golub, et al., SCIENCE, 286: 531-537 (1999); and Slonim, et al., PROCS. OF THE FOURTH ANNUAL INTERNATIONAL CONFERENCE ON COMPUTATIONAL MOLECULAR BIOLOGY, Tokyo, Japan, April 8-11, p 263-272 (2000)), the significance analysis of microarrays (SAM) method (Tusher, et al., PROC. NATL. ACAD. SCI. U.S.A., 98:5116-5121 (2001)), support vector machines, and artificial neural networks. Other rank tests, survival analyses, correlation metrics, or statistical methods can also be used.

The Cox proportional hazards model is the most commonly used regression model for censored survival data. See, for example, Tibshirani, CLINICAL & INVESTIGATIVE MEDICINE, 5:63-68 (1982); Allison, SURVIVAL ANALYSIS USING THE SAS SYSTEM: A PRACTICAL GUIDE (Cary N C: SAS Institute, 1995); and Therneau and Grambsch, MODELING SURVIVAL DATA: EXTENDING THE COX MODEL (New York: Springer, 2000). The Cox model examines the relationship between survival and one or more covariates or predictors. As used herein, the term “survival” is not limited to real death or survival. Instead, the term should be interpreted broadly to cover any time-associated event. The Cox proportional hazards model is often considered more general than many other regression models in that the Cox model is not based on any assumptions concerning the nature or shape of the underlying survival distribution. The Cox model assumes that the underlying hazard rate is a function of independent covariates or predictors, and no assumptions are made about the nature or shape of the hazard function.

A non-limiting example of the Cox proportional hazards model is described by the following equation:

$${H}_{i} ( t ) = {H}_{0} ( t ) exp ( {\sum }_{j = 1}^{k} {β}_{j} {x}_{ij} ) \qquad ( 1 )$$

where i is a subscript for subject, and Hᵢ(t) is the hazard at time t and represents the probability of an endpoint (e.g., death, disease progression, or another time-associated event) at time t, given that the subject has survived up to time t. Xⱼ denotes a predictor or covariate, which can be continuous, dichotomous or other ordered categorical variables. The Cox proportional regression model assumes that the effects of the predictors are constant over time. In many embodiments, Xⱼ represents changes in the expression level of gene j in peripheral blood cells (e.g., PBMCs) of solid tumor patients following initiation of an anti-cancer treatment. Where Xⱼ has a highly skewed distribution, logarithmic transformation can be performed to reduce the effect of extreme values. H₀(t) is the baseline hazard at time t, and designates the hazard for the respective individual when all independent covariates are equal to zero. In a Cox model, the baseline hazard function is unspecified. Despite the lack of a specified baseline hazard function, the Cox model can still be estimated, for example, by the method of partial likelihood.

The Cox model depicted by Equation (1) is semi-parametric because while the baseline hazard can take any form, the coefficients of the covariates are estimated. Consider two observations i and i′ that differ in their x-values, with the corresponding linear predictors

$$\begin{matrix}PI = ( {\sum }_{j = 1}^{k} {β}_{j} {x}_{ij} ) and & ( 2 ) \\ {PI}^{′} = ( {\sum }_{j = 1}^{k} {β}_{j} {x}_{{i}^{′} j} ) & ( 3 )\end{matrix}$$

The ratio of Hᵢ(t) over H_{i′}(t),

$$\begin{matrix}{H}_{i} ( t ) / {H}_{{i}^{′}} ( t ) = [ {H}_{0} ( t ) exp ( PI ) ] / [ {H}_{0} ( t ) exp ( {PI}^{′} ) ] \\ = exp ( PI ) / exp ( {PI}^{′} )\end{matrix} \qquad ( 4 )$$

is independent of time t. Therefore, the Cox model in Equation (1) is a proportional hazards model.

Equation (5) describes a univariate Cox model in which only a single predictor is assessed by Cox regression:

Hᵢ(t)=H₀(t)exp(βXᵢ) (5)

The hazard ratio (RR) is defined as exp(β), which represents the relative risk of an event (e.g., death or disease progression) for one unit change in the predictor. In many applications, PBMC expression values are presented as logarithms of base 2, and a one-unit change corresponds to a doubling of expression. The natural logarithm of the hazard ratio produces coefficient β. Where an S-Plus or R package is utilized, the hazard ratio RR can be generated using the “coxph( )” function in the package.

In the univariate Cox analysis, a hazard ratio of less than 1 indicates a negative coefficient β. As a result, an increase in the value of the predictor produces a reduced instantaneous risk of the event (e.g., death or disease progression). Conversely, a decrease in the value of the predictor produces a greater instantaneous risk of the event. Likewise, a hazard ratio of greater than 1 suggests a positive coefficient β. Therefore, an increase (or decrease) in the value of the predictor produces a greater (or lesser) instantaneous risk of the event.

As a non-limiting example, an increase in predictor Xᵢ, as compared to predictor Xᵢ, produces a lesser PI when coefficient β is negative and, therefore, a lesser Hᵢ(t) compared to H_{i′}(t). See Equations (2), (3) and (4), where k=1. Conversely, a decrease in Xᵢ produces a greater Hᵢ(t) compared to H_{i′}(t). When coefficient β is positive, an increase (or decrease) in Xᵢ produces a greater (or lesser) Hᵢ(t) as compared to H_{i′}(t). Accordingly, the Cox proportional hazards model can be used to evaluate the relative risk of a time-associated event among different individuals.

Once a Cox model is fit, at least three tests of hypothesis can be used to assess the statistical significance of the covariate. These tests are the likelihood ratio test, Wald's test, and the score test. In many embodiments, the p-values determined by one or more of these tests for the correlation between gene expression changes from baseline and patient outcomes are no more than 0.05, 0.01, 0.005, 0.001, 0.0005, 0.0001, or less. The hazard ratio for a prognostic gene of the present invention can be less than 1, such as no more than 0.5, 0.33, 0.25, 0.2, 0.1, or less. The hazard ratio of the gene can also be greater than 1, such as at least 2, 3, 4, 5, 10, or more. A hazard ratio of less than one indicates that an increased expression level of the gene in peripheral blood cells of a solid tumor patient is suggestive of a good prognosis of the patient, while a hazard ratio of greater than 1 suggests that an increased expression level of the gene in peripheral blood cells of the patient is indicative of a poor prognosis of the patient.

The present invention also contemplates the use of multivariate Cox models to correlate peripheral blood gene expression changes and clinical outcomes of solid tumor patients. Each multivariate Cox model includes two or more covariates or predictors, and each covariate represents a change in the expression level of a predictor gene in peripheral blood cells (e.g., PBMCs) of solid tumor patients during the course of an anti-cancer treatment. In many embodiments, the change in the expression level is measured from baseline. Interactions among different covariates can also be introduced into the model.

Predictors that are significant on univariate analyses (e.g., having p-values of no more than 0.05, 0.01, 0.005, 0.001 or less) can be tested in a multivariate model. In one example, predictors are selected for multivariate analysis using forward stepwise selection. For instance, the single most significant predictor on univariate analysis can be first entered into the multivariate model, followed by the next most significant predictor, and so on. In some instances, dimension reduction methods (such as principal component analysis or sliced inverse regression) are used to reduce the number of predictors in a multivariate model potentially without compromising the predictive performance of the model.

Various computer programs are available for carrying out Cox regression analysis. Examples of these programs include, but are not limited to, the S-Plus, SAS, or SPSS packages. See, for instance, Allison, SURVIVAL ANALYSIS USING THE SAS SYSTEM: A PRACTICAL GUIDE (Cary N C: SAS Institute, 1995); and Therneau, A PACKAGE FOR SURVIVAL ANALYSIS IN S (Technical Report, www.mayo.edu/hsr/people/therneau/survival.ps, Mayo Foundation, 1999).

Modified Cox models can also be used. For instance, stratification factors can be introduced into a Cox model to allow for nonproportional hazards to exist between levels of variables. Residuals can be used to discover the correct functional form for a predictor, identify subjects who are poorly predicted by the model, or assess the proportional hazards assumption. In addition, time varying covariates, time dependent coefficients, multiple/correlated observations, or multiple time scales can be analyzed by a modified Cox model. Penalized Cox models or frailty models can also be used.

The present invention also features the use of other correlation or statistical methods for the identification of correlations between peripheral blood gene expression changes and patient outcomes. These methods include, but are not limited to, weighted voting (Golub, et al., SCIENCE, 286:531-537 (1999)), support vector machines (Su, et al., CANCER RESEARCH, 61:7388-93 (2001)), K-nearest neighbors (Ramaswamy, et al., PROCEEDINGS OF THE NATIONAL ACADEMY OF SCIENCES OF THE USA, 98:15149-15154 (2001)), correlation coefficients (van't Veer, et al., NATURE, 415:530-536 (2002)), or other suitable pattern recognition programs.

Examples of solid tumor treatments that can be evaluated according to the present invention include, but are not limited to, drug therapy (e.g., CCI-779 therapy), chemotherapy, hormone therapy, radiotherapy, immunotherapy, surgery, gene therapy, anti-angiogenesis therapy, palliative therapy, or other conventional or non-conventional therapies, or any combination thereof. Solid tumors amenable to the present invention include, without limitation, RCC, prostate cancer, head/neck cancer, ovarian cancer, testicular cancer, brain tumor, breast cancer, lung cancer, colon cancer, pancreas cancer, stomach cancer, bladder cancer, skin cancer, cervical cancer, uterine cancer, liver cancer, or other tumors that do not have their origins in blood or lymph cells. The status or progression of a solid tumor can be evaluated using direct or indirect visualization procedures. Suitable visualization methods include, but are not limited to, scans (such as X-rays, computerized axial tomography (CT), magnetic resonance imaging (MRI), positron emission tomography (PET), or ultrasonography (U/S)), biopsy, palpation, endoscopy, laparoscopy, or other suitable means as appreciated by those skilled in the art. Clinical outcome of a solid tumor can be assessed by numerous criteria. In many embodiments, clinical outcome is measured based on patient response to a therapeutic treatment. Examples of time-associated clinical outcome measures include, but are not limited to, time to disease progression (TTP), time to death (TTD or Survival), time to complete response, time to partial response, time to minor response, time to stable disease, or a combination thereof.

TTP refers to the interval from the date of initiation of a treatment until the first day of measurement of progressive disease. TTD refers to the interval from the date of initiation of a treatment to the time of death. Complete response, partial response, minor response, stable disease or progressive disease can be evaluated, without limitation, using the WHO Reporting Criteria, such as those described in WHO Publication, No. 48 (World Health Organization, Geneva, Switzerland, 1979). Under the Criteria, uni- or bidimensionally measurable lesions are measured at each assessment. When multiple lesions are present in any organ, up to 6 representative lesions can be selected, if available.

In many cases, “complete response” (CR) is defined as complete disappearance of all measurable and evaluable disease, determined by two observations not less than 4 weeks apart. There is no new lesion and no disease related symptom. “Partial response” (PR) in reference to bidimensionally measurable disease means decrease by at least about 50% of the sum of the products of the largest perpendicular diameters of all measurable lesions as determined by 2 observations not less than 4 weeks apart. “Partial response” in reference to unidimensionally measurable disease means decrease by at least about 50% in the sum of the largest diameters of all lesions as determined by 2 observations not less than 4 weeks apart. It is not necessary for all lesions to have regressed to qualify for partial response, but no lesion should have progressed and no new lesion should appear. The assessment should be objective. “Minor response” in reference to bidimensionally measurable disease means about 25% or greater decrease but less than about 50% decrease in the sum of the products of the largest perpendicular diameters of all measurable lesions. “Minor response” in reference to unidimensionally measurable disease means decrease by at least about 25% but less than about 50% in the sum of the largest diameters of all lesions.

“Stable disease” (SD) in reference to bidimensionally measurable disease means less than about 25% decrease or less than about 25% increase in the sum of the products of the largest perpendicular diameters of all measurable lesions. “Stable disease” in reference to unidimensionally measurable disease means less than about 25% decrease or less than about 25% increase in the sum of the diameters of all lesions. No new lesions should appear. “Progressive disease” (PD) refers to a greater than or equal to about a 25% increase in the size of at least one bidimensionally (product of the largest perpendicular diameters) or unidimensionally measurable lesion or appearance of a new lesion. The occurrence of pleural effusion or ascites is also considered as progressive disease if this is substantiated by positive cytology. Pathological fracture or collapse of bone is not necessarily evidence of disease progression.

In one non-limiting example, overall subject tumor response for uni- and bidimensionally measurable disease is determined according to Table 1.

TABLE 1
Overall Subject Tumor Response

| Response in | Response in | |
|---|---|---|
| Bidimensionally | Unidimensionally | Overall Subject |
| Measurable Disease | Measurable Disease | Tumor Response |
| PD | Any | PD |
| Any | PD | PD |
| SD | SD or PR | SD |
| SD | CR | PR |
| PR | SD or PR or CR | PR |
| CR | SD or PR | PR |
| CR | CR | CR |

Overall subject tumor response for non-measurable disease can be assessed, for instance, in the following situations:

a) Overall complete response: if non-measurable disease is present, it should disappear completely. Otherwise, the subject cannot be considered as an “overall complete responder.”

b) Overall progression: in case of a significant increase in the size of non-measurable disease or the appearance of a new lesion, the overall response will be progression.

For the correlation studies, solid tumor patients can be classified based on their respective clinical outcomes. They can also be classified using traditional clinical risk assessment methods. In many cases, these risk assessment methods employ a number of prognostic factors which separate solid tumor patients into different prognosis or risk groups. One example of these methods is the Motzer risk assessment for RCC, as described in Motzer, et al., J CLIN ONCOL, 17:2530-2540 (1999). Patients in different risk groups may have different responses to a therapy.

A variety of types of peripheral blood samples can be used for the identification of correlations between peripheral blood gene expression changes and patient outcomes. Peripheral blood samples suitable for this purpose include, but are not limited to, whole blood samples or samples comprising enriched PBMCs. By “enriched,” it means that the percentage of PBMCs in the sample is higher than that in whole blood. In many cases, the PBMC percentage in an enriched sample is at least 1, 2, 3, 4, 5 or more times higher than that in whole blood. In many other cases, the PBMC percentage in an enriched sample is at least 90%, 95%, 98%, 99%, 99.5%, or more. Blood samples containing enriched PBMCs can be prepared by using any method known in the art, such as Ficoll gradients centrifugation or CPTs (cell purification tubes).

A peripheral blood sample employed in the present invention can be isolated at any time prior to, during or after an anti-cancer treatment. For instance, peripheral blood samples can be isolated prior to a therapeutic treatment. These samples are herein referred to as “baseline” or “pretreatment” samples. Gene expression profiles in these samples are herein referred to as “baseline” or “pretreatment” profiles. For another instance, peripheral blood samples can be isolated from solid tumor patients at 1, 2, 3, 4, 5, 6, 7, 8, 9, 10, 11, 12, 13, 14, 15, or 16 weeks following initiation of an anti-cancer treatment. Other time intervals can also be used for the preparation of blood samples.

In many embodiments, gene expression changes are determined by measuring alterations between gene expression profiles at a specified time after initiation of an anti-cancer treatment and baseline expression profiles. Reference time points other than baseline can also be used.

Peripheral blood gene expression changes can be evaluated using global gene expression analysis. Methods suitable for this purpose include, but are not limited to, nucleic acid arrays (such as cDNA or oligonucleotide arrays), protein arrays, 2-dimensional SDS-polyacrylamide gel electrophoresis/mass spectrometry, and other high throughput nucleotide or polypeptide detection techniques.

Nucleic acid arrays allow for quantitative detection of the expression levels of a large number of genes at one time. Examples of nucleic acid arrays include, but are not limited to, Genechip® microarrays from Affymetrix (Santa Clara, Calif.), cDNA microarrays from Agilent Technologies (Palo Alto, Calif.), and bead arrays described in U.S. Pat. Nos. 6,288,220 and 6,391,562.

The polynucleotides to be hybridized to a nucleic acid array can be labeled with one or more labeling moieties to allow for detection of hybridized polynucleotide complexes. The labeling moieties can include compositions that are detectable by spectroscopic, photochemical, biochemical, bioelectronic, immunochemical, electrical, optical or chemical means. Exemplary labeling moieties include radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like. Unlabeled polynucleotides can also be employed. The polynucleotides can be DNA, RNA, or a modified form thereof.

Hybridization reactions can be performed in absolute or differential hybridization formats. In the absolute hybridization format, polynucleotides prepared from one sample, such as a peripheral blood sample isolated from a solid tumor patient at a specific time during the course of an anti-cancer treatment, are hybridized to a nucleic acid array. Signals detected after the formation of hybridization complexes indicate the polynucleotide levels in the sample. In the differential hybridization format, polynucleotides prepared from two biological samples, such as one from a patient of interest and the other from a reference patient, are labeled with different labeling moieties. A mixture of these differently labeled polynucleotides is added to a nucleic acid array. The nucleic acid array is then examined under conditions in which the emissions from the different labels are individually detectable. In one embodiment, the fluorophores Cy3 and Cy5 (Amersham Pharmacia Biotech, Piscataway N.J.) are used as the labeling moieties for the differential hybridization format.

Signals gathered from a nucleic acid array can be analyzed using commercially available software, such as those provided by Affymetrix or Agilent Technologies. Controls, such as for scan sensitivity, probe labeling and cDNA/cRNA quantitation, can be included in the hybridization experiments. In many embodiments, the nucleic acid array expression signals are scaled or normalized before being subject to further analysis. For instance, the expression signals for each gene can be normalized to take into account variations in hybridization intensities when more than one array is used under similar test conditions. Signals for individual polynucleotide complex hybridization can also be normalized using the intensities derived from internal normalization controls contained on each array. In addition, genes with relatively consistent expression levels across the samples can be used to normalize the expression levels of other genes. In one embodiment, the expression levels of the genes are normalized across the samples such that the mean is zero and the standard deviation is one. In another embodiment, the expression data detected by nucleic acid arrays are subject to a variation filter which excludes genes showing minimal or insignificant variation across all samples.

### II. IDENTIFICATION OF RCC PROGNOSTIC GENES

RCC comprises the majority of all cases of kidney cancer and is one of the ten most common cancers in industrialized countries. The five-year survival rate for advanced RCC is less than 5 percent. RCC is usually detected by imaging methods, and 30 percent of apparently non-metastatic patients undergo relapse after surgery and eventually succumb to disease. Recent expression profiling studies have demonstrated that the transcriptional profiles of primary malignancies are radically altered from the transcriptional profiles of the corresponding normal tissue (for a review see Slonim, PHARMACOGENOMICS, 2:123-136 (2001)). Specific microarray studies examining RCC tumor transcriptional profiles in detail (Young, et al., AM. J. PATHOL., 158:1639-1651 (2001)) have identified many classes of genes altered between normal kidney tissue and primary RCC tumors.

Several prognostic factors and scoring indices have been developed for patients diagnosed with RCC, typified by multivariate assessments of several key indicators. One example is the Motzer risk assessment scores, which employ five prognostic factors proposed by Motzer, et al., J CLIN ONCOL, 17:2530-2540 (1999)-namely, Karnofsky performance status, serum lactate dehydrognease, hemoglobin, serum calcium, and presence/absence of prior nephrectomy. RCC patients can be classified into favorable, intermediate or poor prognosis based on their respective Motzer risk assessment scores.

The present invention features surrogate gene markers for prognosis of RCC. The expression levels of these genes in peripheral blood cells of RCC patients change during the course of a CCI-779 therapy, and the magnitudes of these changes from baseline expression levels are correlated with a continuous measure of clinical outcome, such as TTP or TTD.

CCI-779 is a small molecule inhibitor of the mTOR pathway that is currently undergoing evaluation as a cytostatic agent in the various indications in the field of oncology and in such indications as multiple sclerosis. CCI-779 is an ester analog of the immunosuppressant rapamycin and as such is a potent, selective inhibitor of the mammalian target of rapamycin. The mammalian target of rapamycin (mTOR) activates multiple signaling pathways, including phosphorylation of p70s6kinase, which results in increased translation of 5′ TOP mRNAs encoding proteins involved in translation and entry into the G1 phase of the cell cycle. By virtue of its inhibitory effects on mTOR and cell cycle control, CCI-779 functions as a cytostatic and immunosuppressive agent.

111 advanced RCC patients (34 females and 77 males) were treated with 25, 75, or 250 mg of CCI-779 intravenous (IV) infusion once weekly until evidence of disease progression. Gene expression results of a subset of 45 patients (18 females and 27 males) were further analyzed. RCC tumors of these 45 patients were classified at the clinical sites as conventional (clear cell) carcinomas (24), granular (1), papillary (3), or mixed subtypes (7). Ten tumors were classified as unknown. RCC patients were primarily of Caucasian descent (44 Caucasian, 1 African-American) and had a mean age of 58 years (range of 40-78 years). Inclusion criteria included patients with histologically confirmed advanced renal cancer who had received prior therapy for advanced disease, or who had not received prior therapy for advanced disease but were not appropriate candidates to receive high doses of IL-2 therapy. Other inclusion criteria included patients with (1) bi-dimensionally measurable evidence of disease; (2) evidence of progression of the disease prior to study entry; (3) an age of 18 years or older; (4) ANC>1500/μL, platelet>100,000/μL and hemoglobin>8.5 g/dL; (5) adequate renal function evidenced by serum creatinine<1.5× upper limit of normal; (6) adequate hepatic function evidenced by bilirubin<1.5× upper limit of normal and AST<3× upper limit of normal (or AST<5× upper limit of normal if liver metastases were present); (7) serum cholesterol<350 mg/dL, triglycerides<300 mg/dL; (8) ECOG performance status 0-1; and (9) a life expectancy of at least 12 weeks. Exclusion criteria included patients who had (1) the presence of known CNS metastases; (2) surgery or radiotherapy within 3 weeks of start of dosing; (3) chemotherapy or biologic therapy for RCC within 4 weeks of start of dosing; (4) treatment with a prior investigational agent within 4 weeks of start of dosing; (5) immunocompromised status including those known to be HIV positive, or receiving concurrent use of immunosuppressive agents including corticosteroids; (6) active infections; (7) required treatment with anticonvulsant therapy; (8) presence of unstable angina/myocardial infarction within 6 months/ongoing treatment of life-threatening arrythmia; (9) history of prior malignancy in past 3 years; (10) hypersensitivity to macrolide antibiotics; and (11) pregnancy or any other illness which would substantially increase the risk associated with participation in the study. The selected RCC patients were treated with one of 3 doses of CCI-779 (25 mg, 75 mg, or 250 mg) administered as a 30 minute IV infusion once weekly for the duration of the trial.

Clinical staging and size of residual, recurrent or metastatic disease were recorded prior to treatment and every 8 weeks following initiation of CCI-779 therapy. Tumor size was measured in centimeters and reported as the product of the longest diameter and its perpendicular. Measurable disease was defined as any bidimensionally measurable lesion where both diameters>1.0 cm by CT-scan, X-ray or palpation. Tumor response was determined by the sum of the products of all measurable lesions. The categories for assignment of clinical response were given by the clinical protocol definitions (i.e., progressive disease, stable disease, minor response, partial response, and complete response). The category for assignment of prognosis under the Motzer risk assessment (favorable vs intermediate vs poor) was also used. Among the 45 RCC patients, 6 were assigned a favorable risk assessment, 17 patients possessed an intermediate risk score, and 22 patients received a poor prognosis classification. In addition to the categorical classifications, overall survival and time to disease progression were also monitored as clinical endpoints.

PBMCs were isolated from peripheral blood of the RCC patients prior to CCI-779 therapy and every 8 weeks after initiation of the treatment. Nucleic acid samples were prepared from the isolated PBMCs and hybridized to HG-U95A genechips (Affymetrix, Santa Clara, Calif.) according to the manufacturer's guideline. See GeneChip® Expression Analysis—Technical Manual (Part No. 701021 Rev. 1, Affymetrix, Inc. 1999-2001), the entire content of which is incorporated herein by reference. Signals were calculated from probe intensities by the MAS 4 algorithm, and signal intensities were converted to frequencies using the scale frequency normalization method as described in the Examples.

To identify specific alterations in transcript levels in PBMCs that were correlated with patient outcome, a Cox proportional hazards regression was employed, which accounts for the effect of censoring of clinical outcome measures, to model outcome as a function of log₂-transformed expression levels (in units of ppm). Cox regression analyses were performed on two clinical outcome measures—TTP and TTD—for each of the 5,469 qualifiers that passed the initial filtering criteria (at least 1 “present” call across the data set, and at least one transcript with a frequency of >10 ppm; see Example 3). In the Cox proportional hazard analysis the hazard ratio associated with each transcript indicates the likelihood of a favorable or non-favorable outcome, where a hazard ratio of less than 1 indicates less risk for increasing levels of the covariate and a hazard ratio of greater than 1 indicates higher risk.

For each transcript and outcome measure, hazard ratios were calculated and the Wald p-value for the hypothesis that the hazard ratio was equal to 1 (i.e., no risk) was calculated. The number of tests that were nominally significant out of the 5,469 tests performed for each outcome measure was calculated for five Type I (i.e., false-positive) error levels. To adjust for the fact that the 5,469 tests were not independent, a permutation-based approach was then employed to evaluate how often the observed number of significant tests would be found under the null hypothesis of no risk.

Cox proportional hazard regression models were fit to assess the association between gene expression levels measured by HG-U95A Affymetrix microarrays and clinical outcome. Models were fit using expression levels from each of 5,469 qualifiers that passed the initial filtering criteria in the baseline, 8 week, and 16 week samples (at least 1 “present” call across the samples, and at least one transcript with a frequency of >10 ppm). Two clinical measures—TTD and TTP—were tested for their association with change from baseline scaled frequency. Change from baseline was calculated based on log₂-transformed scaled frequency values, and was computed for 8 weeks and for 16 weeks after baseline.

The results of comparisons of clinical outcomes with change from baseline expression levels are summarized in Tables 2A and 2B for change at 8 weeks, and in Tables 3A and 3B for change at 16 weeks. The evidence for association between clinical outcomes and change from baseline gene expression is strong for both outcome variables at 16 weeks.

TABLE 2A
Permutation Results for Cox Proportional Hazards Regressions of
Clinical Outcome of TTD on 8-Week Change from Baseline
Log₂-Transformed Frequencies (n = 30 patients)
Time to Death

| | | Percentage of Permutations |
|---|---|---|
| | | for which Number of |
| | Observed Number of | Nominally Significant Cox |
| | Nominally Significant | Regressions Equals or |
| α-Confidence Level | Cox Regressions* | Exceeds Observed Number |
| 0.1 | 584 | 44% (220/500) |
| 0.05 | 295 | 41% (206/500) |
| 0.01 | 46 | 45% (226/500) |
| 0.005 | 25 | 38% (190/500) |
| 0.001 | 5 | 19% (154/500) |

*for 5,469 genes (filtered by “at least one Present call and at least one frequency >10 ppm”)

TABLE 2B
Permutation Results for Cox Proportional Hazards Regressions of
Clinical Outcome of TTP on 8-Week Change from Baseline
Log₂-Transformed Frequencies (n = 30 patients)
Time to Progression

| | | Percentage of Permutations |
|---|---|---|
| | | for which Number of |
| | Observed Number of | Nominally Significant Cox |
| | Nominally Significant | Regressions Equals or |
| α-Confidence Level | Cox Regressions* | Exceeds Observed Number |
| 0.1 | 901 | 11% (53/500) |
| 0.05 | 503 | 10% (51/500) |
| 0.01 | 95 | 16% (79/500) |
| 0.005 | 47 | 16% (78/500) |
| 0.001 | 2 | 61% (308/500) |

*for 5,469 genes (filtered by “at least one Present call and at least one frequency >10 ppm”)

TABLE 3A
Permutation Results for Cox Proportional Hazards Regressions of
Clinical Outcome of TTD on 16-Week Change from Baseline
Log₂-Transformed Frequencies (n = 22 patients)
Time to Death

| | | Percentage of Permutations |
|---|---|---|
| | | for which Number of |
| | Observed Number of | Nominally Significant Cox |
| | Nominally Significant | Regressions Equals or |
| α-Confidence Level | Cox Regressions* | Exceeds Observed Number |
| 0.1 | 1106 | 3.8% (19/500) |
| 0.05 | 646 | 3.6% (18/500) |
| 0.01 | 173 | 2.2% (11/500) |
| 0.005 | 80 | 4.2% (21/500) |
| 0.001 | 14 | 4.0% (20/500) |

*for 5,469 genes (filtered by “at least one Present call and at least one frequency >10 ppm”)

TABLE 3B
Permutation Results for Cox Proportional Hazards Regressions of
Clinical Outcome of TTP on 16-Week Change from Baseline
Log₂-Transformed Frequencies (n = 22 patients)
Time to Progression

| | | Percentage of Permutations |
|---|---|---|
| | | for which Number of |
| | Observed Number of | Nominally Significant Cox |
| | Nominally Significant | Regressions Equals or |
| α-Confidence Level | Cox Regressions* | Exceeds Observed Number |
| 0.1 | 1317 | 1.2% (6/500) |
| 0.05 | 872 | 0.4% (2/500) |
| 0.01 | 283 | 0.4% (2/500) |
| 0.005 | 136 | 0.4% (2/500) |
| 0.001 | 15 | 3.4% (17/500) |

*for 5,469 genes (filtered by “at least one Present call and at least one frequency >10 ppm”)

Tables 4A and 4B provide 20 exemplary genes in PBMCs with changes in transcript levels at 16 weeks that were correlated with low risk (hazard ratio<1.0) or high risk (hazard ratio>1.0) for TTP, respectively. Tables 5A and 5B list 20 exemplary genes in PBMCs with changes in transcript levels at 16 weeks that were correlated with low risk (hazard ratio<1.0) or high risk (hazard ratio>1.0) for TTD, respectively. Table 6 provides annotations of these genes.

TABLE 4A
20 Exemplary Genes in RCC PBMCs of CCI-779 Treated Patients
Exhibiting Changes at 16 Weeks Significantly Correlated with TTP
(Elevated Expression at 16 Weeks Suggests Good Prognosis
for Progression)

| Qualifier | Hazard Ratio | P-Value | Gene Name | Unigene ID |
|---|---|---|---|---|
| 36131_at | 0.0805 | 0.0056 | UNK_AJ012008 | Hs.74276 |
| 935_at | 0.1098 | 0.0013 | CAP | Hs.104125 |
| 40441_g_at | 0.1186 | 0.0016 | DKFZP564M2423 | Hs.165998 |
| 37007_at | 0.1250 | 0.0055 | TDE1 | Hs.272168 |
| 410_s_at | 0.1345 | 0.0054 | CSNK2B | Hs.165843 |
| 33666_at | 0.1501 | 0.0109 | HNRPC | Hs.182447 |
| 32234_at | 0.1502 | 0.0119 | DYT1 | Hs.19261 |
| 41185_f_at | 0.1523 | 0.0169 | SMT3H2 | Hs.180139 |
| 32594_at | 0.1561 | 0.0092 | CCT4 | Hs.79150 |
| 40063_at | 0.1562 | 0.0006 | NDP52 | Hs.154230 |
| 36585_at | 0.1584 | 0.0047 | ARF4 | Hs.75290 |
| 34849_at | 0.1747 | 0.0055 | SARS | Hs.4888 |
| 37023_at | 0.1763 | 0.0223 | LCP1 | Hs.16488 |
| 39342_at | 0.1763 | 0.0046 | MARS | Hs.279946 |
| 38943_at | 0.1764 | 0.0050 | HCCS | Hs.211571 |
| 590_at | 0.1765 | 0.0024 | ICAM2 | Hs.347326 |
| 35787_at | 0.1833 | 0.0004 | UNK_AI986201 | Hs.355812 |
| 41551_at | 0.1891 | 0.0015 | RER1 | Hs.40500 |
| 37738_g_at | 0.1973 | 0.0014 | PCMT1 | Hs.79137 |
| 36950_at | 0.1978 | 0.0380 | UNK_X90872 | Hs.279929 |

TABLE 4B
20 Exemplary Genes in RCC PBMCs of CCI-779 Treated Patients
Exhibiting Changes at 16 Weeks Significantly Correlated with TTP
(Elevated Expression at 16 Weeks Suggests Poor Prognosis
for Progression)

| Qualifier | Hazard Ratio | P-Value | Gene Name | Unigene ID |
|---|---|---|---|---|
| 41833_at | 70.3014 | 0.0022 | JTB | Hs.6396 |
| 38590_r_at | 34.3415 | 0.0013 | PTMA | Hs.250655 |
| 41231_f_at | 25.2728 | 0.0124 | HMG17 | |
| 34392_s_at | 20.1103 | 0.0027 | DKFZP564B163 | Hs.3642 |
| 35298_at | 14.9081 | 0.0202 | EIF3S7 | Hs.55682 |
| 36637_at | 13.3407 | 0.0152 | ANXA11 | Hs.75510 |
| 36198_at | 13.1169 | 0.0004 | KIAA0016 | Hs.75187 |
| 33619_at | 12.3924 | 0.0225 | RPS13 | Hs.165590 |
| 32205_at | 12.0630 | 0.0016 | PRKRA | Hs.18571 |
| 36587_at | 11.8495 | 0.0223 | EEF2 | Hs.75309 |
| 38738_at | 11.0671 | 0.0028 | SMT3H1 | Hs.85119 |
| 36186_at | 10.9675 | 0.0016 | RNPS1 | Hs.75104 |
| 40874_at | 10.7873 | 0.0085 | EDF1 | Hs.174050 |
| 40203_at | 9.7115 | 0.0031 | SUI1 | Hs.150580 |
| 41834_g_at | 9.5538 | 0.0123 | JTB | Hs.6396 |
| 39415_at | 9.3960 | 0.0133 | HNRPK | Hs.129548 |
| 34647_at | 8.1524 | 0.0164 | DDX5 | Hs.76053 |
| 36515_at | 8.1450 | 0.0002 | GNE | Hs.5920 |
| 41235_at | 8.0415 | 0.0011 | ATF4 | Hs.181243 |
| 37912_at | 7.9835 | 0.0026 | TRAF4 | Hs.8375 |

TABLE 5A
20 Exemplary Genes in RCC PBMCs OF CCI-779 Treated Patients
Exhibiting Changes at 16 Weeks Significantly Correlated With TTD
(Elevated Expression at 16 Weeks Suggests Good Prognosis
for Survival)

| | Hazard | | | Unigene |
|---|---|---|---|---|
| Qualifier | Ratio | P-Value | Gene Name | ID |
| 35770_at | 0.0568 | 0.0034 | ATP6S1 | Hs.6551 |
| 40771_at | 0.0811 | 0.0313 | MSN | Hs.170328 |
| 1394_at | 0.1206 | 0.0856 | UNK_L25080 | Hs.77273 |
| 33659_at | 0.1228 | 0.0152 | CFL1 | Hs.180370 |
| 39738_at | 0.1243 | 0.0083 | APOL | |
| 1878_g_at | 0.1327 | 0.0115 | ERCC1 | Hs.59544 |
| 1863_s_at | 0.1379 | 0.0569 | UNK_U67092 | Hs.194382 |
| 39092_at | 0.1671 | 0.0162 | PURB | Hs.301005 |
| AFFX- | 0.1832 | 0.0242 | BACTIN3_Hs_AFFX | Hs.288061 |
| HSAC07/ | | | | |
| X00351_3_at | | | | |
| 32318_s_at | 0.1943 | 0.0673 | ACTB | Hs.288061 |
| 41332_at | 0.1978 | 0.0002 | POLR2E | Hs.24301 |
| 37023_at | 0.2310 | 0.0320 | LCP1 | Hs.16488 |
| 39354_at | 0.2387 | 0.0034 | KIAA0106 | Hs.120 |
| 36666_at | 0.2499 | 0.0082 | P4HB | Hs.75655 |
| 33424_at | 0.2521 | 0.0005 | RPN1 | Hs.2280 |
| 36581_at | 0.2542 | 0.0554 | GARS | Hs.283108 |
| 36668_at | 0.2676 | 0.0458 | DIA1 | Hs.274464 |
| 691_g_at | 0.2699 | 0.0382 | P4HB | Hs.75655 |
| 40768_s_at | 0.2769 | 0.0473 | NUP214 | Hs.170285 |
| 41421_at | 0.2885 | 0.0472 | KIAA0909 | Hs.107362 |

TABLE 5B
20 Exemplary Genes in RCC PBMCs OF CCI-779 Treated Patients
Exhibiting Changes at 16 Weeks Significantly Correlated With TTD
(Elevated Expression at 16 Weeks Suggests Poor Prognosis
for Survival)

| Qualifier | Hazard Ratio | P-Value | Gene Name | Unigene ID |
|---|---|---|---|---|
| 39739_at | 29.9466 | 0.0023 | MYH9 | Hs.32916 |
| 33215_g_at | 19.6111 | 0.0050 | RPMS12 | Hs.9964 |
| 34401_at | 18.4364 | 0.0088 | UQCRFS1 | Hs.3712 |
| 36765_at | 17.0062 | 0.0001 | DKFZP434I114 | Hs.72620 |
| 41190_at | 15.5344 | 0.0082 | TNFRSF12 | Hs.180338 |
| 1817_at | 14.8747 | 0.0066 | PFDN5 | Hs.288856 |
| 34570_at | 13.6770 | 0.0011 | RPS27A | Hs.3297 |
| 31708_at | 12.3739 | 0.0055 | RPL30 | Hs.334807 |
| 34608_at | 12.1813 | 0.0164 | GNB2L1 | Hs.5662 |
| 121_at | 11.8726 | 0.0040 | PAX8 | Hs.73149 |
| 34646_at | 11.7518 | 0.0007 | RPS7 | Hs.301547 |
| 327_f_at | 11.7018 | 0.0206 | RPS20 | |
| 41553_at | 11.5948 | 0.0015 | C8ORF1 | Hs.40539 |
| 36333_at | 11.3559 | 0.0218 | RPL7 | Hs.153 |
| 1683_at | 11.2771 | 0.0001 | WIT-1 | |
| 32341_f_at | 10.8460 | 0.0088 | RPL23A | Hs.350046 |
| 324_f_at | 10.8113 | 0.0089 | BTF3 | |
| 162_at | 10.7452 | 0.0058 | USP11 | Hs.171501 |
| 32435_at | 10.5153 | 0.0145 | RPL19 | Hs.252723 |
| 32432_f_at | 9.6275 | 0.0239 | RPL15 | Hs.74267 |

TABLE 6
Annotations of RCC Prognostic genes

| | Accession No. | |
|---|---|---|
| Qualifier | (Entrez) | Gene Title |
| 36131_at | AJ012008 | Homo sapiens genes encoding RNCC |
| | | protein, DDAH protein, Ly6-C protein, Ly6- |
| | | D protein and immunoglobulin receptor |
| 935_at | L12168 | adenylyl cyclase-associated protein |
| 40441_g_at | AL080119 | DKFZP564M2423 protein |
| 37007_at | U49188 | tumor differentially expressed 1 |
| 410_s_at | X57152 | casein kinase 2, beta polypeptide |
| 33666_at | M16342 | heterogeneous nuclear ribonucleoprotein C |
| | | (C1/C2) |
| 32234_at | AF007871 | dystonia 1, torsion (autosomal dominant; |
| | | torsin A) |
| 41185_f_at | AI971724 | SMT3 (suppressor of mif two 3, yeast) |
| | | homolog 2 |
| 32594_at | AF026291 | chaperonin containing TCP1, subunit 4 |
| | | (delta) |
| 40063_at | U22897 | nuclear domain 10 protein |
| 36585_at | M36341 | ADP-ribosylation factor 4 |
| 34849_at | X91257 | seryl-tRNA synthetase |
| 37023_at | J02923 | lymphocyte cytosolic protein 1 (L-plastin) |
| 39342_at | X94754 | methionine-tRNA synthetase |
| 38943_at | U36787 | holocytochrome c synthase (cytochrome c |
| | | heme-lyase) |
| 590_at | M32334 | intercellular adhesion molecule 2 |
| 35787_at | AI986201 | ESTs, Moderately similar to cytoplasmic |
| | | dynein intermediate chain 1 [H. sapiens] |
| 41551_at | AW044624 | similar to S. cerevisiae RER1 |
| 37738_g_at | D25547 | protein-L-isoaspartate (D-aspartate) O- |
| | | methyltransferase |
| 36950_at | X90872 | H. sapiens mRNA for gp25L2 protein |
| 41833_at | AB016492 | jumping translocation breakpoint |
| 38590_r_at | M14630 | prothymosin, alpha (gene sequence 28) |
| 41231_f_at | X13546 | high-mobility group (nonhistone |
| | | chromosomal) protein 17 |
| 34392_s_at | AL050268 | DKFZP564B163 protein |
| 35298_at | U54558 | eukaryotic translation initiation factor 3, |
| | | subunit 7 (zeta, 66/67 kD) |
| 36637_at | L19605 | annexin A11 |
| 36198_at | D13641 | translocase of outer mitochondrial |
| | | membrane 20 (yeast) homolog |
| 33619_at | L01124 | ribosomal protein S13 |
| 32205_at | AF072860 | protein kinase, interferon-inducible double |
| | | stranded RNA dependent activator |
| 36587_at | Z11692 | eukaryotic translation elongation factor 2 |
| 38738_at | X99584 | SMT3 (suppressor of mif two 3, yeast) |
| | | homolog 1 |
| 36186_at | L37368 | RNA-binding protein S1, serine-rich domain |
| 40874_at | AJ005259 | endothelial differentiation-related factor 1 |
| 40203_at | AJ012375 | putative translation initiation factor |
| 41834_g_at | AB016492 | jumping translocation breakpoint |
| 39415_at | X72727 | heterogeneous nuclear ribonucleoprotein K |
| 34647_at | X52104 | DEAD/H (Asp-Glu-Ala-Asp/His) box |
| | | polypeptide 5 (RNA helicase, 68 kD) |
| 36515_at | AJ238764 | UDP-N-acetylglucosamine-2-epimerase/N- |
| | | acetylmannosamine kinase |
| 41235_at | AL022312 | activating transcription factor 4 (tax- |
| | | responsive enhancer element B67) |
| 37912_at | X80200 | TNF receptor-associated factor 4 |
| 35770_at | D16469 | ATPase, H+ transporting, lysosomal |
| | | (vacuolar proton pump), subunit 1 |
| 40771_at | Z98946 | moesin |
| 1394_at | L25080 | Homo sapiens GTP-binding protein (rhoA) |
| | | mRNA, complete cds. |
| 33659_at | X95404 | cofilin 1 (non-muscle) |
| 39738_at | Z82215 | apolipoprotein L |
| 1878_g_at | M13194 | excision repair cross-complementing rodent |
| | | repair deficiency, complementation group 1 |
| | | (includes overlapping antisense sequence) |
| 1863_s_at | U67092 | Cluster Incl U67092: Human ataxia- |
| | | telangiectasia locus protein (ATM) gene, |
| | | exons 1a, 1b, 2, 3 and 4, partial cds. |
| 39092_at | AW007731 | purine-rich element binding protein B |
| AFFX- | X00351 | BACTIN3 control sequence (H. sapiens) |
| HSAC07/X00351_3_at | | [AFFX] |
| 32318_s_at | X63432 | actin, beta |
| 41332_at | D38251 | polymerase (RNA) II (DNA directed) |
| | | polypeptide E (25 kD) |
| 37023_at | J02923 | lymphocyte cytosolic protein 1 (L-plastin) |
| 39354_at | D14662 | anti-oxidant protein 2 (non-selenium |
| | | glutathione peroxidase, acidic calcium- |
| | | independent phospholipase A2) |
| 36666_at | M22806 | procollagen-proline, 2-oxoglutarate 4- |
| | | dioxygenase (proline 4-hydroxylase), beta |
| | | polypeptide (protein disulfide isomerase; |
| | | thyroid hormone binding protein p55) |
| 33424_at | Y00281 | ribophorin I |
| 36581_at | U09510 | glycyl-tRNA synthetase |
| 36668_at | M28713 | diaphorase (NADH) (cytochrome b-5 |
| | | reductase) |
| 691_g_at | J02783 | procollagen-proline, 2-oxoglutarate 4- |
| | | dioxygenase (proline 4-hydroxylase), beta |
| | | polypeptide (protein disulfide isomerase; |
| | | thyroid hormone binding protein p55) |
| 40768_s_at | X64228 | nucleoporin 214 kD (CAIN) |
| 41421_at | AB020716 | KIAA0909 protein |
| 39739_at | AF054187 | myosin, heavy polypeptide 9, non-muscle |
| 33215_g_at | Y11681 | ribosomal protein, mitochondrial, S12 |
| 34401_at | L32977 | ubiquinol-cytochrome c reductase, Rieske |
| | | iron-sulfur polypeptide 1 |
| 36765_at | AL080154 | DKFZP434I114 protein |
| 41190_at | U83598 | tumor necrosis factor receptor superfamily, |
| | | member 12 (translocating chain-association |
| | | membrane protein) |
| 1817_at | D89667 | prefoldin 5 |
| 34570_at | S79522 | ribosomal protein S27a |
| 31708_at | L05095 | ribosomal protein L30 |
| 34608_at | M24194 | guanine nucleotide binding protein (G |
| | | protein), beta polypeptide 2-like 1 |
| 121_at | X69699 | paired box gene 8 |
| 34646_at | Z25749 | ribosomal protein S7 |
| 327_f_at | L06498 | ribosomal protein S20 |
| 41553_at | AI738702 | chromosome 8 open reading frame 1 |
| 36333_at | X57958 | ribosomal protein L7 |
| 1683_at | X69950 | Wilms tumor associated protein |
| 32341_f_at | U37230 | ribosomal protein L23a |
| 324_f_at | X53281 | basic transcription factor 3 |
| 162_at | U44839 | ubiquitin specific protease 11 |
| 32435_at | X63527 | ribosomal protein L19 |
| 32432_f_at | L25899 | ribosomal protein L15 |

Each qualifier in Tables 4A, 4B, 5A and 5B represents an oligonucleotide probe set on the HG-U95A genechip. The RNA transcript(s) of a gene identified by the qualifier can hybridize under nucleic acid array hybridization conditions to at least one oligonucleotide probe (PM or perfect match probe) of the qualifier. Preferably, the RNA transcript(s) of the gene does not hybridize under nucleic acid array hybridization conditions to the mismatch probe (MM) of the PM probe. An MM probe is identical to the corresponding PM probe except for a single, homomeric substitution at or near the center of the mismatch probe. For a 25-mer PM probe, the MM probe has a homomeric base change at the 13th position.

In many cases, the RNA transcript(s) of a gene identified by a qualifier can hybridize under nucleic acid array hybridization conditions to at least 50%, 60%, 70%, 80%, 90% or 100% of the PM probes of that qualifier, but not to their corresponding MM probes. In many other cases, the discrimination score (R) for each of these PM probes, as measured by the ratio of the hybridization intensity difference of the corresponding probe pair (i.e., PM−MM) over the overall hybridization intensity (i.e., PM+MM), is at least 0.015, 0.02, 0.05, 0.1, 0.2, 0.3, 0.4, 0.5 or greater. In still many other cases, the RNA transcript(s) of a gene identified by a qualifier can produce a “present” call under the default settings of a genechip, e.g., the threshold Tau is 0.015 and the significance level α₁ is 0.4. See GeneChip® Expression Analysis—Data Analysis Fundamentals (Part No. 701190 Rev. 2, Affymetrix, Inc., 2002), the entire content of which is incorporated herein by reference.

The sequence of each PM probe on the HG-U95A genechip, and the corresponding target sequence from which the PM probe is derived, can be obtained from Affymetrix's sequence databases. See, for example, www.affymetrix.com/support/technical/byproduct.affx?product=hgu133. All of these PM probe sequences and their corresponding target sequences are incorporated herein by reference.

Each gene listed in Tables 4A, 4B, 5A and 5B, and the corresponding unigene ID and Entrez accession number, were identified according to HG-U95A genechip annotation. A unigene is composed of a non-redundant set of gene-oriented clusters. Each unigene cluster is believed to include sequences that represent a unique gene. Additional information for the genes listed in Tables 4A, 4B, 5A and 5B can be obtained from the Entrez database at National Center for Biotechnology Information (NCBI) (Bethesda, Md.) based on their corresponding unigene IDs or Entrez accession numbers.

Gene(s) identified by a HG-U95A qualifier can also be determined by BLAST searching the target sequence of the qualifier against a human genome sequence database. Human genome sequence databases suitable for this purpose include, but are not limited to, the NCBI human genome database. NCBI provides BLAST programs, such as “blastn,” for searching its sequence databases. In one embodiment, BLAST search of the NCBI human genome database is carried out by using an unambiguous segment (e.g., the longest unambiguous segment) of the target sequence of a qualifier. Gene(s) represented by the qualifier is identified as those that have significant sequence identity to the unambiguous segment. In many cases, the identified gene(s) has at least 95%, 96%, 97%, 98%, 99%, or more sequence identity to the unambiguous segment.

As used herein, genes represented by the qualifiers in Tables 4A, 4B, 5A and 5B include not only those that are explicitly described therein, but also those that are not listed in the tables, but nonetheless are capable of hybridizing to the PM probes of the qualifiers in the tables. All of these genes can be used as biological markers for prognosis of RCC or other solid tumors.

The above-described analysis used a Cox proportional hazards regression to identify changes in transcript levels in PBMCs of RCC patients at 8 or 16 weeks (from baseline levels) that are correlated with the continuous measures of clinical outcomes TTP and TTD. Permutation analyses indicated that there were significant associations between changes at 16 weeks and the clinical outcomes of TTP and TTD, but less significant associations between PBMC transcriptional changes at 8 weeks and these clinical outcomes.

The finding that transcriptional changes in PBMCs appear to “lag behind” CCI-779 exposure is of great interest, since it supports the theory that transcriptional alterations in PBMCs following CCI-779 therapy reflect the response of circulating cells of peripheral blood to changes in the tumor, rather than direct transcriptional alterations by CCI-779 in the blood. This theory explains the observation that changes in PBMC transcript levels at 16 weeks were more significantly correlated with clinical outcomes, since there can be a lag between achievement of steady state levels of CCI-779 in the blood and responses of PBMCs to changes in the tumor. Thus, the transcripts identified according to the present invention can be used as early pharmacogenomic indicators for drug efficacy. It should be noted that in the majority of transcripts the direction of its significant association with clinical outcome at 16 weeks was identical at 8 weeks but less significant, suggesting that transcriptional patterns in PBMCs at 8 weeks were displaying a similar trend, but not yet as significantly associated with the clinical outcomes of interest as those at 16 weeks.

Of the transcripts that displayed elevations which were significantly negatively associated with disease progression (i.e., PBMC transcripts where increasing elevations in expression at 16 weeks were correlated with increasingly shorter TTPs in RCC patients), there were several observations of interest. Two separate sequences homologous to a jumping translocation breakpoint-encoded transcript were elevated in PBMCs from patients with shorter TTP. In addition, three of the 20 exemplary transcripts negatively associated with disease progression (Table 4B) encoded factors involved in eukaryotic translation initiation and elongation. The identification of these eukaryotic translation associated factors is of interest, since CCI-779 by virtue of its inhibition of the mTOR pathway ultimately represses mammalian translation.

Jumping translocation breakpoint protein JTB was strongly elevated at 16 weeks in PBMC profiles from patients with rapid times to progression. The normal protein encodes a highly conserved membrane transporter protein, which upon the phenomenon of jumping translocation results in a truncated protein lacking the trans-membrane domain (Hatakeyama, et al., ONCOGENE, 18:2085-2090 (1999)). Two separate qualifiers corresponding to this transcript (41833_at and 41834_g_at in Table 4B) were identified among the 20 transcripts where elevations at 16 weeks were significantly associated with rapid disease progression. This finding suggests that overall genomic instability in these patients can be present in the surrogate tissue of PBMCs, since it is unlikely that expression levels measured in the PBMCs of RCC patients reflect any transcripts derived from metastatic renal cancer cells circulating in the blood (Twine, et al., CANCER RES., 63:6069-6075 (2003)).

With respect to survival, a large number of transcripts encoding ribosomal proteins were elevated in patients with shorter times to death. Expression levels of transcripts encoding ribosomal proteins were shown to be strongly correlated with lymphocyte content in several studies (data not shown). Because lymphocytes are not differentially distributed between patients with short versus longer TTP (data not shown), it implies that transcriptional activation in circulating lymphocytes after about 4 months of therapy may bode poorly for the overall survival in RCC patients. Thus, a circulating lymphocyte response can be used to indicate a poor prognosis in RCC patients.

Genes predictive of other time-associated clinical events can also be identified using probe arrays in combination with Cox proportional hazards models. The changes in expression levels of these genes in peripheral blood cells of solid tumor patients during the course of an anti-cancer treatment are statistically significantly correlated with patient outcomes.

### III. PROGNOSIS OF RCC OR OTHER SOLID TUMORS

The present invention features prognostic genes whose expression profile changes in PBMCs are associated with clinical outcomes of solid tumor patients. These prognostic genes can be used as surrogate markers for prognosis of RCC or other solid tumors. They can also be used as pharmacogenomic indicators for the efficacy of CCI-779 or other anti-cancer drugs.

Examples of clinical endpoints that can be assessed by the present invention include, but are not limited to, death, disease progression, or other time-associated events. Suitable measures for these clinical endpoints include TTP, TTD, or other time-dependent clinical measures. Any solid tumor or anti-cancer treatment can be evaluated according to the present invention.

In one aspect, the prognosis of a patient of interest involves the following steps:

detecting a change in expression levels of one or more prognostic genes in peripheral blood cells (e.g., PBMCs) of the patient of interest following initiation of an anti-cancer treatment; and

comparing the detected change to a reference change.

Each of the prognostic genes has an altered expression level following initiation of the anti-cancer treatment, and the magnitude of this alteration in PBMCs of patients who have the same solid tumor and receive the same treatment as the patient of interest is correlated with clinical outcome of these patients. As a consequence, the detected change in the patient of interest is predictive of the clinical outcome of the patient.

The gene expression change in a patient of interest can be measured from any reference point, and expression level changes measured from that point in patients who have the same solid tumor are correlated with clinical outcomes of these patients under an appropriate correlation model (e.g., a Cox model or a class-based correlation metric, such as the nearest-neighbor analysis). In many embodiments, the expression level change of a prognostic gene in a patient of interest is determined by measuring the alteration between the expression level of the gene in the peripheral blood of the patient of interest at a specified time following initiation of an anti-cancer treatment and the baseline expression level of the prognostic gene.

The specified time used for determining gene expression changes in a patient of interest can be selected such that significant correlation exists between the changes measured at that time and patient outcomes under a permutation analysis. The permutation analysis evaluates how often the observed number of significant tests would be found under the null hypothesis of no risk. In one example, the specified time is selected such that the percentage of permutations for which number of nominally significant correlations equals or exceeds the observed number is below 10%, 5%, 1%, 0.5% or less at a predetermined α-confidence level (e.g., 0.05, 0.01, 0.005 or less). In a non-limiting example, the specified time is at least 16 weeks after initiation of an anti-cancer treatment. Times less than 16 weeks, such as about 1, 2, 3, 4, 5, 6, 7, 8, 9, 10, 11, 12, 13, 14 or 15 weeks after initiation of an anti-cancer treatment, can also be used.

In many embodiments, the reference change used for the prognosis of a patient of interest is a gene expression change in a reference patient. The reference patient has the same solid tumor and receives the same anti-cancer treatment as the patient of interest. The reference patient can also be a “virtual” patient utilized by a Cox proportional hazard model or another correlation model. The reference change can be determined using the same or comparable methodologies as that for the patient of interest. A difference between the change in the patient of interest and the reference change is suggestive of a relative prognosis of the patient of interest as compared to the reference patient. The reference change and the change in the patient of interest can be determined concurrently or sequentially.

In one embodiment, both the patient of interest and the reference patient have RCC, and both patients receive the same anti-cancer treatment (e.g., a CCI-779 therapy). The gene expression changes in the patient of interest and the reference patient are determined by measuring alterations between expression levels of one or more prognostic genes in peripheral blood cells of the respective patient at a specified time (e.g., 16 weeks) following initiation of the treatment and the baseline expression levels of the prognostic gene(s). The magnitudes of these alterations in PBMCs of RCC patients who receive the same anti-cancer treatment are correlated with clinical outcomes of these patients under a Cox proportional hazards model.

Where a prognostic gene has a hazard ratio of greater than 1, a greater change in the expression level of the gene in peripheral blood cells of the patient of interest, as compared to that in the reference patient, is indicative of a poorer prognosis for the patient of interest compared to the reference patient. Conversely, a lesser change in the patient of interest is indicative of a better prognosis for the patient of interest compared to the reference patient.

Where a prognostic gene has a hazard ratio of less than 1, a greater change in the expression level of the gene in peripheral blood cells of the patient of interest, as compared to that in the reference patient, is indicative of a better prognosis for the patient of interest. A lesser change in the patient of interest is indicative of a poorer prognosis for the patient of interest.

Prognostic genes suitable for this purpose include, but are not limited to, those depicted in Tables 4A, 4B, 5A and 5B. Genes selected from Tables 4A and 4B can be used to assess the relative TTP of a patient of interest, while genes selected from Tables 5A and 5B can be used to evaluate the relative TTD of a patient of interest.

Other prognostic genes can also be used. In many embodiments, each prognostic gene employed in the present invention shows a statistically significant correlation between expression level changes in PBMCs of RCC patients following initiation of an anti-cancer treatment (e.g., a CCI-779 therapy) and clinical outcomes of these patients. In many instances, the p-value of this correlation is no more than 0.05, 0.01, 0.005, 0.001, 0.0005, 0.0001, or less. The hazard ratio for a prognostic gene can be no more than 0.5, 0.33, 0.25, 0.2, 0.1 or less. The hazard ratio can also be at least 2, 3, 4, 5, 10, or more.

In many other embodiments, the reference change used for the prognosis of a patient of interest has an empirically or experimentally determined value. A patient of interest is considered to have a poor or good prognosis if the expression level change in the patient of interest is above or below the empirically or experimentally determined value. For instance, where a prognostic gene has a hazard ratio of less than 1 (or greater than 1), the observation that the change in the expression level of the gene in peripheral blood cells of the patient of interest from baseline is above the empirically determined value is predictive of a good (or poor) prognosis of the patient of interest.

In one embodiment, the empirically or experimentally determined value represents an average change between expression levels of a prognostic gene in peripheral blood cells (e.g., PBMCs) of reference patients at a specified time after initiation of an anti-cancer treatment and baseline expression levels. Suitable averaging methods for this purpose include, but are not limited to, arithmetic means, harmonic means, average of absolute values, average of log-transformed values, or weighted average. The reference patients have the same solid tumor and receive the same treatment as the patient of interest. In many cases, the references patients are composed of patients who have similar prognoses (e.g., good, intermediate, or poor prognoses).

The present invention features the use of univariate or multivariate Cox models for the prognosis of a patient of interest. The univariate Cox analysis (e.g., Equation (5)) provides the relative risk of a time-associated event (e.g., death or disease progression) for one unit change in one predictor. In many embodiments, the predictor represents changes in the expression level of a prognostic gene in peripheral blood cells of solid tumor patients following initiation of an anti-cancer treatment. As described above, one can choose to partition a patient of interest into different prognosis groups at a threshold value, where patients with expression level changes above the threshold have higher risk, and patients with expression level changes below the threshold have lower risk, or vice versa, depending on whether the gene is an indicator of bad (RR>1) or good (RR<1) prognosis. In addition, model fitting can provide an estimate for the baseline hazard H₀(t) or the coefficient β, thereby enabling a more quantitative assessment of the clinical outcome of a patient of interest. Prognostic genes identified by the univariate Cox analysis can be used individually, or in combination, for the prognosis of a patient of interest. In a multivariate Cox model (e.g., Equation (1)), the linear predictor PI can be used as a risk index for the prognosis of a patient of interest. In many instances, a multivariate Cox model can be built by stepwise entry of each individual gene into the model, where the first gene entered is pre-selected from those genes having significant univariate p-values, and the gene selected for entry into the model at each subsequent step is the gene that best improves the fit of the model to the data.

The distribution of risk index values can be calculated in a training set to determine an appropriate cut-point to distinguish high and low risk. A continuum of cut-points can be examined. Using the risk index function and the high/low risk cut-point estimated in the training set, the risk index value for each test case can be calculated and used to assign a patient of interest to a high or low risk group.

In many embodiments, the accuracy of predicting the clinical outcome of a patient of interest (i.e., the ratio of correct calls over the total of correct and incorrect calls) is at least 50%, 60%, 70%, 80%, 90%, or more. The effectiveness of clinical outcome prediction can also be measured by sensitivity and specificity. In many embodiments, the sensitivity and specificity of a prognostic gene employed in the present invention is at least 50%, 60%, 70%, 80%, 90%, 95%, or more. Moreover, the peripheral blood-based prognosis can be combined with other clinical evidence to improve the accuracy of the eventual clinical outcome prediction.

A variety of types of blood samples can be used to determine gene expression changes in a patient of interest or the reference patient(s). Examples of blood samples suitable for this purpose include, but are not limited to, whole blood samples or samples comprising enriched PBMCs. Other blood samples can also be used, and statistically significant correlations exist between patient outcomes and gene expression changes in these blood samples.

Numerous methods are available for detecting gene expression levels in a blood sample of interest. For instance, the expression level of a gene can be determined by measuring the level of the RNA transcript(s) of the gene. Suitable methods for this purpose include, but are not limited to, quantitative RT-PCT, Northern Blot, in situ hybridization, slot-blotting, nuclease protection assays, or nucleic acid arrays (including bead arrays). The expression level of a gene can also be determined by measuring the level of the polypeptide(s) encoded by the gene. Suitable methods for this purpose include, but are not limited to, immunoassays (such as ELISA, RIA, FACS, or Western Blot), 2-dimensional gel electrophoresis, mass spectrometry, or protein arrays.

In one aspect, the expression level of a prognostic gene is determined by measuring the RNA transcript level of the gene in a peripheral blood sample. RNA can be isolated from the peripheral blood sample using a variety of methods. Exemplary methods include guanidine isothiocyanate/acidic phenol method, the TRIZOL® Reagent (Invitrogen), or the Micro-FastTrack™ 2.0 or FastTrack™ 2.0 mRNA Isolation Kits (Invitrogen). The isolated RNA can be either total RNA or mRNA. The isolated RNA can be amplified to cDNA or cRNA before subsequent detection or quantitation. The amplification can be either specific or non-specific. Suitable amplification methods include, but are not limited to, reverse transcriptase PCR (RT-PCR), isothermal amplification, ligase chain reaction, and Qbeta replicase.

In one embodiment, the amplification protocol employs reverse transcriptase. The isolated mRNA can be reverse transcribed into cDNA using a reverse transcriptase, and a primer consisting of oligo d(T) and a sequence encoding the phage T7 promoter. The cDNA thus produced is single-stranded. The second strand of the cDNA is synthesized using a DNA polymerase, combined with an RNase to break up the DNA/RNA hybrid. After synthesis of the double-stranded cDNA, T7 RNA polymerase is added, and cRNA is then transcribed from the second strand of the doubled-stranded cDNA. The amplified cDNA or cRNA can be detected or quantitated by hybridization to labeled probes. The cDNA or cRNA can also be labeled during the amplification process and then detected or quantitated.

In another embodiment, quantitative RT-PCR (such as TaqMan, ABI) is used for detecting or comparing the RNA transcript level of a prognostic gene of interest. Quantitative RT-PCR involves reverse transcription (RT) of RNA to cDNA followed by relative quantitative PCR (RT-PCR).

In PCR, the number of molecules of the amplified target DNA increases by a factor approaching two with every cycle of the reaction until some reagent becomes limiting. Thereafter, the rate of amplification becomes increasingly diminished until there is not an increase in the amplified target between cycles. If a graph is plotted on which the cycle number is on the X axis and the log of the concentration of the amplified target DNA is on the Y axis, a curved line of characteristic shape can be formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR is proportional to the starting concentration of the target before the PCR is begun. By determining the concentration of the PCR products of the target DNA in PCR reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived may be determined for the respective tissues or cells. This direct proportionality between the concentration of the PCR products and the relative mRNA abundances is true in the linear range portion of the PCR reaction.

The final concentration of the target DNA in the plateau portion of the curve is determined by the availability of reagents in the reaction mix and is independent of the original concentration of target DNA. Therefore, in one embodiment, the sampling and quantifying of the amplified PCR products are carried out when the PCR reactions are in the linear portion of their curves. In addition, relative concentrations of the amplifiable cDNAs can be normalized to some independent standard, which may be based on either internally existing RNA species or externally introduced RNA species. The abundance of a particular mRNA species may also be determined relative to the average abundance of all mRNA species in the sample.

In one embodiment, the PCR amplification utilizes internal PCR standards that are approximately as abundant as the target. This strategy is effective if the products of the PCR amplifications are sampled during their linear phases. If the products are sampled when the reactions are approaching the plateau phase, then the less abundant product may become relatively over-represented. Comparisons of relative abundances made for many different RNA samples, such as is the case when examining RNA samples for differential expression, may become distorted in such a way as to make differences in relative abundances of RNAs appear less than they actually are. This can be improved if the internal standard is much more abundant than the target. If the internal standard is more abundant than the target, then direct linear comparisons may be made between RNA samples.

A problem inherent in clinical samples is that they are of variable quantity or quality. This problem can be overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

In another embodiment, the relative quantitative RT-PCR uses an external standard protocol. Under this protocol, the PCR products are sampled in the linear portion of their amplification curves. The number of PCR cycles that are optimal for sampling can be empirically determined for each target cDNA fragment. In addition, the reverse transcriptase products of each RNA population isolated from the various samples can be normalized for equal concentrations of amplifiable cDNAs. While empirical determination of the linear range of the amplification curve and normalization of cDNA preparations are tedious and time-consuming processes, the resulting RT-PCR assays may, in certain cases, be superior to those derived from a relative quantitative RT-PCR with an internal standard.

In yet another embodiment, nucleic acid arrays (including bead arrays) are used for detecting or comparing the expression profiles of a prognostic gene of interest. The nucleic acid arrays can be commercial oligonucleotide or cDNA arrays. They can also be custom arrays comprising concentrated probes for the prognostic genes of the present invention. In many examples, at least 15%, 20%, 25%, 30%, 35%, 40%, 45%, 50%, or more of the total probes on a custom array of the present invention are probes for RCC or other solid tumor prognostic genes. These probes can hybridize under stringent or nucleic acid array hybridization conditions to the RNA transcripts, or the complements thereof, of the corresponding prognostic genes.

As used herein, “stringent conditions” are at least as stringent as, for example, conditions G-L shown in Table 6. “Highly stringent conditions” are at least as stringent as conditions A-F shown in Table 6. Hybridization is carried out under the hybridization conditions (Hybridization Temperature and Buffer) for about four hours, followed by two 20-minute washes under the corresponding wash conditions (Wash Temp. and Buffer).

TABLE 6
Stringency Conditions

| | Poly- | | | |
|---|---|---|---|---|
| Stringency | nucleotide | Hybrid | Hybridization | Wash Temp. |
| Condition | Hybrid | Length (bp)¹ | Temperature and Buffer^{H} | and Buffer^{H} |
| A | DNA:DNA | >50 | 65° C.; 1xSSC -or- | 65° C.; 0.3xSSC |
| | | | 42° C.; 1xSSC, 50% formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% formamide | |
| F | RNA:RNA | <50 | T_{F}*; 1xSSC | T_{f}*; 1xSSC |
| G | DNA:DNA | >50 | 65° C.; 4xSSC -or- | 65° C.; 1xSSC |
| | | | 42° C.; 4xSSC, 50% formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% formamide | |
| L | RNA:RNA | <50 | T_{L}*; 2xSSC | T_{L}*; 2xSSC |

¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity.
^{H}SSPE (1x SSPE is 0.15M NaCl, 10 mM NaH₂PO₄, and 1.25 mM EDTA, pH 7.4) can be substituted for SSC (1x SSC is 0.15M NaCl and 15 mM sodium citrate) in the hybridization and wash buffers.
T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base pairs in length, Tₘ(° C.) = 81.5 + 16.6(log₁₀[Na⁺]) + 0.41(% G + C) − (600/N), where N is the number of bases in the hybrid, and [Na⁺] is the molar concentration of sodium ions in the hybridization buffer ([Na⁺] for 1x SSC = 0.165 M).

In one example, a nucleic acid array of the present invention includes at least 2, 5, 10, or more different probes. Each of these probes is capable of hybridizing under stringent or nucleic acid array hybridization conditions to a different respective prognostic gene of the present invention (e.g., genes selected from Tables 4A, 4B, 5A and B). Multiple probes for the same prognostic gene can be used. The probe density on a nucleic acid array can be in any range.

The probes for a prognostic gene of the present invention can be DNA, RNA, PNA, or a modified form thereof. The nucleotide residues in each probe can be either naturally occurring residues (such as deoxyadenylate, deoxycytidylate, deoxyguanylate, deoxythymidylate, adenylate, cytidylate, guanylate, and uridylate), or synthetically produced analogs that are capable of forming desired base-pair relationships. Examples of these analogs include, but are not limited to, aza and deaza pyrimidine analogs, aza and deaza purine analogs, and other heterocyclic base analogs, wherein one or more of the carbon and nitrogen atoms of the purine and pyrimidine rings are substituted by heteroatoms, such as oxygen, sulfur, selenium, and phosphorus. Similarly, the polynucleotide backbones of the probes can be either naturally occurring (such as through 5′ to 3′ linkage), or modified. For instance, the nucleotide units can be connected via non-typical linkage, such as 5′ to 2′ linkage, so long as the linkage does not interfere with hybridization. For another instance, peptide nucleic acids, in which the constitute bases are joined by peptide bonds rather than phosphodiester linkages, can be used.

The probes for the prognostic genes can be stably attached to discrete regions on a nucleic acid array. By “stably attached,” it means that a probe maintains its position relative to the attached discrete region during hybridization and signal detection. The position of each discrete region on the nucleic acid array can be either known or determinable. Any method known in the art can be used to make the nucleic acid arrays of the present invention.

In another embodiment, nuclease protection assays are used to quantitate RNA transcript levels in peripheral blood samples. There are many different versions of nuclease protection assays. The common characteristic of these nuclease protection assays is that they involve hybridization of an antisense nucleic acid with the RNA to be quantified. The resulting hybrid double-stranded molecule is then digested with a nuclease that digests single-stranded nucleic acids more efficiently than double-stranded molecules. The amount of antisense nucleic acid that survives digestion is a measure of the amount of the target RNA species to be quantified. Examples of suitable nuclease protection assays include the RNase protection assay provided by Ambion, Inc. (Austin, Tex.).

Hybridization probes or amplification primers for the prognostic genes of the present invention can be prepared by using any method known in the art. For prognostic genes whose genomic locations have not been determined or whose identities are solely based on EST or mRNA data, the probes/primers for these genes can be derived from the target sequences of the corresponding qualifiers, or the corresponding EST or mRNA sequences.

In one embodiment, the probes/primers for a prognostic gene significantly diverge from the sequences of other prognostic genes. This can be achieved by checking potential probe/primer sequences against a human genome sequence database, such as the Entrez database at the NCBI. One algorithm suitable for this purpose is the BLAST algorithm. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. The initial neighborhood word hits act as seeds for initiating searches to find longer HSPs containing them. The word hits are then extended in both directions along each sequence to increase the cumulative alignment score. Cumulative scores are calculated using, for nucleotide sequences, the parameters M (reward score for a pair of matching residues; always >0) and N (penalty score for mismatching residues; always <0). The BLAST algorithm parameters W, T, and X determine the sensitivity and speed of the alignment. These parameters can be adjusted for different purposes, as appreciated by those skilled in the art.

In another aspect, the expression levels of the prognostic genes of the present invention are determined by measuring the levels of polypeptides encoded by the prognostic genes. Methods suitable for this purpose include, but are not limited to, immunoassays such as ELISA, RIA, FACS, dot blot, Western Blot, immunohistochemistry, and antibody-based radioimaging. In addition, high-throughput protein sequencing, 2-dimensional SDS-polyacrylamide gel electrophoresis, mass spectrometry, or protein arrays can be used.

In one embodiment, ELISAs are used for detecting the levels of the target proteins. In an exemplifying ELISA, antibodies capable of binding to the target proteins are immobilized onto selected surfaces exhibiting protein affinity, such as wells in a polystyrene or polyvinylchloride microtiter plate. Samples to be tested are then added to the wells. After binding and washing to remove non-specifically bound immunocomplexes, the bound antigen(s) can be detected. Detection can be achieved by the addition of a second antibody which is specific for the target proteins and is linked to a detectable label. Detection can also be achieved by the addition of a second antibody, followed by the addition of a third antibody that has binding affinity for the second antibody, with the third antibody being linked to a detectable label. Before being added to the microtiter plate, cells in the samples can be lysed or extracted to separate the target proteins from potentially interfering substances.

In another exemplifying ELISA, the samples suspected of containing the target proteins are immobilized onto the well surface and then contacted with the antibodies. After binding and washing to remove non-specifically bound immunocomplexes, the bound antigen is detected. Where the initial antibodies are linked to a detectable label, the immunocomplexes can be detected directly. The immunocomplexes can also be detected using a second antibody that has binding affinity for the first antibody, with the second antibody being linked to a detectable label.

Another exemplary ELISA involves the use of antibody competition in the detection. In this ELISA, the target proteins are immobilized on the well surface. The labeled antibodies are added to the well, allowed to bind to the target proteins, and detected by means of their labels. The amount of the target proteins in an unknown sample is then determined by mixing the sample with the labeled antibodies before or during incubation with coated wells. The presence of the target proteins in the unknown sample acts to reduce the amount of antibody available for binding to the well and thus reduces the ultimate signal.

Different ELISA formats can have certain features in common, such as coating, incubating or binding, washing to remove non-specifically bound species, and detecting the bound immunocomplexes. For instance, in coating a plate with either antigen or antibody, the wells of the plate can be incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test samples. Examples of these nonspecific proteins include bovine serum albumin (BSA), casein and solutions of milk powder. The coating allows for blocking of nonspecific adsorption sites on the immobilizing surface and thus reduces the background caused by nonspecific binding of antisera onto the surface.

In ELISAs, a secondary or tertiary detection means can be used. After binding of a protein or antibody to the well, coating with a non-reactive material to reduce background, and washing to remove unbound material, the immobilizing surface is contacted with the control or clinical or biological sample to be tested under conditions effective to allow immunocomplex (antigen/antibody) formation. These conditions may include, for example, diluting the antigens and antibodies with solutions such as BSA, bovine gamma globulin (BGG) and phosphate buffered saline (PBS)/Tween and incubating the antibodies and antigens at room temperature for about 1 to 4 hours or at 4° C. overnight. Detection of the immunocomplex is facilitated by using a labeled secondary binding ligand or antibody, or a secondary binding ligand or antibody in conjunction with a labeled tertiary antibody or third binding ligand.

Following all incubation steps in an ELISA, the contacted surface can be washed so as to remove non-complexed material. For instance, the surface may be washed with a solution such as PBS/Tween, or borate buffer. Following the formation of specific immunocomplexes between the test sample and the originally bound material, and subsequent washing, the occurrence of the amount of immunocomplexes can be determined.

To provide a detecting means, the second or third antibody can have an associated label to allow detection. In one embodiment, the label is an enzyme that generates color development upon incubating with an appropriate chromogenic substrate. Thus, for example, one may contact and incubate the first or second immunocomplex with a urease, glucose oxidase, alkaline phosphatase or hydrogen peroxidase-conjugated antibody for a period of time and under conditions that favor the development of further immunocomplex formation (e.g., incubation for 2 hours at room temperature in a PBS-containing solution such as PBS-Tween).

After incubation with the labeled antibody, and subsequent washing to remove unbound material, the amount of label can be quantified, e.g., by incubation with a chromogenic substrate such as urea and bromocresol purple or 2,2′-azido-di-(3-ethyl)-benzthiazoline-6-sulfonic acid (ABTS) and H₂O₂, in the case of peroxidase as the enzyme label. Quantitation can be achieved by measuring the degree of color generation, e.g., using a spectrophotometer.

Another method suitable for detecting polypeptide levels is RIA (radioimmunoassay). An exemplary RIA is based on the competition between radiolabeled-polypeptides and unlabeled polypeptides for binding to a limited quantity of antibodies. Suitable radiolabels include, but are not limited to, I¹²⁵. In one embodiment, a fixed concentration of I¹²⁵-labeled polypeptide is incubated with a series of dilution of an antibody specific to the polypeptide. When the unlabeled polypeptide is added to the system, the amount of the I¹²⁵-polypeptide that binds to the antibody is decreased. A standard curve can therefore be constructed to represent the amount of antibody-bound I¹²⁵-polypeptide as a function of the concentration of the unlabeled polypeptide. From this standard curve, the concentration of the polypeptide in unknown samples can be determined. Protocols for conducting RIA are well known in the art.

Suitable antibodies for the present invention include, but are not limited to, polyclonal antibodies, monoclonal antibodies, chimeric antibodies, humanized antibodies, single chain antibodies, Fab fragments, or fragments produced by a Fab expression library. Neutralizing antibodies (i.e., those which inhibit dimer formation) can also be used. Methods for preparing these antibodies are well known in the art. In one embodiment, the antibodies of the present invention can bind to the corresponding prognostic gene products or other desired antigens with binding affinities of at least 10⁴ M⁻¹, 10⁵ M⁻¹, 10⁶ M⁻¹, 10⁷ M⁻¹, or more.

The antibodies of the present invention can be labeled with one or more detectable moieties to allow for detection of antibody-antigen complexes. The detectable moieties can include compositions detectable by spectroscopic, enzymatic, photochemical, biochemical, bioelectronic, immunochemical, electrical, optical or chemical means. The detectable moieties include, but are not limited to, radioisotopes, chemiluminescent compounds, labeled binding proteins, heavy metal atoms, spectroscopic markers such as fluorescent markers and dyes, magnetic labels, linked enzymes, mass spectrometry tags, spin labels, electron transfer donors and acceptors, and the like.

The antibodies of the present invention can be used as probes to construct protein arrays for the detection of expression profiles of the prognostic genes. Methods for making protein arrays or biochips are well known in the art. In many embodiments, a substantial portion of probes on a protein array of the present invention are antibodies specific for the prognostic gene products. For instance, at least 10%, 20%, 30%, 40%, 50%, or more probes on the protein array can be antibodies specific for the prognostic gene products.

In yet another aspect, the expression levels of the prognostic genes are determined by measuring the biological functions or activities of these genes. Where a biological function or activity of a prognostic gene is known, suitable in vitro or in vivo assays can be developed to evaluate this function or activity. These assays can be subsequently used to assess the level of expression of the prognostic gene.

Gene expression levels employed in the present invention can be absolute, normalized, or relative levels. Suitable normalization procedures include, but are not limited to, those used in the conventional nucleic acid array analysis or those described in Hill, et al., GENOME BIOL, 2:research0055.1-0055.13 (2001). In one example, the expression levels are normalized such that the mean is zero and the standard deviation is one. In another example, the expression levels are normalized based on internal or external controls. In still another example, the expression levels are normalized against one or more control transcripts with known abundances in blood samples. In many embodiments, the expression levels used for assessing gene expression changes in a patient of interest and the reference patient(s) are determined using the same or comparable methodologies.

The present invention also features electronic systems useful for prognosis of RCC or other solid tumors. These systems include input or computing devices for receiving or calculating gene expression changes in a solid tumor patient of interest and the reference expression changes. The reference expression changes can also be stored in a database or another medium, and are retrievable by the electronic systems of the present invention. The comparison between the gene expression changes in the patient of interest and the reference expression changes can be conduced electronically, such as by a processor or computer. In many embodiments, the systems also include or are capable of downloading from another source (e.g., an internet server) one or more programs, such as a Cox model, a k-nearest-neighbors analysis, or a weighted voting algorithm. These programs can be used to compare the gene expression changes in the patient of interest to the reference changes, or to correlate gene expression changes in solid tumor patients to clinical outcomes of these patients. In one example, an electronic system of the present invention is coupled to a nucleic acid array to receive or process the expression data generated from the array.

In still another aspect, the present invention provides kits useful for prognosis of RCC or other solid tumors. Each kit includes or consists essentially of at least one probe for an RCC or solid tumor prognostic gene (e.g., a gene selected from Tables 4A, 4B, 5A or 5B). Reagents or buffers that facilitate the use of the kit can also be included. Any type of probe can be using in the present invention, such as hybridization probes, amplification primers, antibodies, or other high-affinity binders.

In one embodiment, a kit of the present invention includes or consists essentially of at least 1, 2, 3, 4, 5, 6, 7, 8, 9, 10, or more polynucleotide probes or primers. Each probe/primer can hybridize under stringent or nucleic acid array hybridization conditions to a different solid tumor prognostic gene, such as those selected from Tables 4A, 4B, 5A or 5B. As used herein, a polynucleotide can hybridize to a gene if the polynucleotide can hybridize to an RNA transcript, or the complement thereof, of the gene.

In another embodiment, a kit of the present invention includes or consists essentially of one or more antibodies, each of which is capable of binding to a polypeptide encoded by a different solid tumor prognostic gene, such as those selected from Tables 4A, 4B, 5A or 5B.

The probes employed in the present invention can be either labeled or unlabeled. Labeled probes can be detectable by spectroscopic, photochemical, biochemical, bioelectronic, immunochemical, electrical, optical, chemical, or other suitable means. Exemplary labeling moieties for a probe include radioisotopes, chemiluminescent compounds, labeled binding proteins, heavy metal atoms, spectroscopic markers, such as fluorescent markers and dyes, magnetic labels, linked enzymes, mass spectrometry tags, spin labels, electron transfer donors and acceptors, and the like.

The kits of the present invention can also have containers containing buffer(s) or reporter-means. In addition, the kits can include reagents for conducting positive or negative controls. In one embodiment, the probes employed in the present invention are stably attached to one or more substrate supports. Nucleic acid hybridization or immunoassays can be directly carried out on the substrate support(s). Suitable substrate supports for this purpose include, but are not limited to, glasses, silica, ceramics, nylons, quartz wafers, gels, metals, papers, beads, tubes, fibers, films, membranes, column matrixes, or microtiter plate wells. In many embodiments, at least 5%, 10%, 20%, 30%, 40%, 50% or more of the total probes in a kit of the present invention are probes for solid tumor prognostic genes.

In another aspect, the present invention features methods of using logistic regression, ANOVA (analysis of variance), ANCOVA (analysis of covariance), MANOVA (multiple analysis of variance), or other correlation or statistical methods for prognosis of a solid tumor in a patient of interest. These methods comprise:

detecting the expression level of at least one solid tumor prognostic gene in peripheral blood cells of the patient of interest at a specified time during the course of an anti-cancer treatment; and

entering the expression level into a correlation or statistical model to determine the prognosis of the patient of interest.

The correlation or statistical model defines a statistically significant correlation between the expression levels of the solid tumor prognostic gene(s) in PBMCs of patients who have the same solid tumor and receive the same treatment as the patient of interest, and clinical outcomes of these patients. In many examples, the correlation or statistical model is capable of producing a qualitative prediction of the clinical outcome of the patient of interest (e.g., good or poor prognosis). Statistical models or analyses suitable for this purpose include, but are not limited to, logistic regression or class-based correlation metrics. In many other examples, the correlation or statistical model is capable of producing a quantitative prediction of the clinical outcome of the patient of interest (e.g., an estimated TTD or TTP). Statistical models or analyses suitable for this purpose include, but are not limited to, a variety of regression, ANOVA or ANCOVA models.

The expression levels used for building the correlation/statistical model or prognosticating the patient of interest can be relative expression levels measured from baseline or another specified reference time point after initiation of the treatment of the corresponding patient. Absolute expression levels can also be used for building the correlation/statistical model or prognosticating the patient of interest. In the latter case, expression levels at baseline or another specified reference time can be used as covariates in the prediction model.

### IV. EVALUATION OF EFFICACY OF ANTI-CANCER TREATMENT

The present invention allows for personalized treatment of RCC or other solid tumors. A patient of interest can be prognosticated during the course of an anti-cancer treatment. A good prognosis indicates that the treatment can be continued, while a poor prognosis suggests that the treatment may be stopped and a different approach should be used to treat the patient. This analysis helps patients avoid unnecessary adverse reactions. It also provides improved safety and increased benefit/risk ratio for the treatment.

In one embodiment, an RCC patient of interest is prognosticated during the course of a CCI-779 therapy. Prognostic genes suitable for this purpose include, but are not limited to, those depicted in Tables 4A, 4B, 5A and 5B. Changes in the expression levels of these prognostic genes in peripheral blood cells of the patient of interest can be determined by using RT-PCR, ELISAs, nucleic acid arrays, protein arrays, protein functional assays or other suitable means. These changes are compared to reference changes to determine the prognosis of the patient of interest. A good prognosis indicates suitability of the CCI-779 treatment for the RCC patient of interest.

Any type of anti-cancer treatment can be evaluated by the present invention. In one non-limiting example, the anti-cancer treatment is a drug therapy. Examples of anti-cancer drugs include, but are not limited to, cytokines, such as interferon or interleukin 2, and chemotherapy drugs, such as CCI-779, AN-238, vinblastine, floxuridine, 5-fluorouracil, or tamoxifen. AN238 is a cytotoxic agent which has 2-pyrrolinodoxorubicin linked to a somatostatin (SST) carrier octapeptide. AN238 can be targeted to SST receptors on the surface of RCC tumor cells. Chemotherapy drugs can be used individually or in combination with other drugs, cytokines, or therapies. In addition, monoclonal antibodies, antiangiogenesis drugs, or anti-growth factor drugs can also be used to treat RCC or other solid tumors.

An anti-cancer treatment can also be surgical. Suitable surgical choices for RCC include, but are not limited to, radical nephrectomy, partial nephrectomy, removal of metastases, arterial embolization, laparoscopic nephrectomy, cryoablation, and nephron-sparing surgery. Moreover, radiation, gene therapy, immunotherapy, adoptive immunotherapy, or other conventional or experimental therapies can be used to treat solid tumors.

It should be understood that the above-described embodiments and the following examples are given by way of illustration, not limitation. Various changes and modifications within the scope of the present invention will become apparent to those skilled in the art from the present description.

### V. EXAMPLES

### Example 1

### Purification of PBMCs and RNA

Whole blood was collected from RCC patients prior to initiation of CCI-779 therapy and following 8 or 16 weeks of therapy. The blood samples were drawn into CPT Cell Preparation Vacutainer Tubes (Becton Dickinson). For each sample, the target volume was 8 ml. PBMCs were isolated over Ficoll gradients according to the manufacturer's protocol (Becton Dickinson). PBMC pellets were stored at −80° C. until samples were processed for RNA.

RNA purification was performed using QIA shredders and Qiagen Rneasy® mini-kits. Samples were harvested in RLT lysis buffer (Qiagen, Valencia, Calif., USA) containing 0.1% beta-mercaptoethanol and processed for total RNA isolation using the RNeasy mini kit (Qiagen, Valencia, Calif., USA). Eluted RNA was quantified using a 96 well plate UV reader monitoring A260/280. RNA qualities (bands for 18S and 28S) were checked by agarose gel electrophoresis in 2% agarose gels. The remaining RNA was stored at −80° C. until processed for Affymetrix genechip hybridization.

### Example 2

### RNA Amplification and Generation of GeneChip Hybridization Probes

Labeled target for oligonucleotide arrays was prepared using a modification of the procedure described in Lockhart, et al., NATURE BIOTECHNOLOGY, 14:1675-1680 (1996). Two micrograms of total RNA were converted to cDNA using an oligo-d(T)24 primer containing a T7 DNA polymerase promoter at the 5′ end. The cDNA was used as the template for in vitro transcription using a T7 DNA polymerase kit (Ambion, Woodlands, Tex., USA) and biotinylated CTP and UTP (Enzo, Farmingdale, N.Y., USA). Labeled CRNA was fragmented in 40 mM Tris-acetate pH 8.0, 100 mM KOAc, 30 mM MgOAc for 35 min at 94° C. in a final volume of 40 mL. Ten micrograms of labeled target were diluted in 1×MES buffer with 100 mg/mL herring sperm DNA and 50 mg/mL acetylated BSA. To normalize arrays to each other and to estimate the sensitivity of the oligonucleotide arrays, in vitro synthesized transcripts of 11 bacterial genes were included in each hybridization reaction as described in Hill, et al., GENOME BIOL., 2:research0055.1-0055.13 (2001). The abundance of these transcripts ranged from 1:300000 (3 ppm) to 1:1000 (1000 ppm) stated in terms of the number of control transcripts per total transcripts. As determined by the signal response from these control transcripts, the sensitivity of detection of the arrays ranged between 2.33 and 4.5 copies per million.

Labeled sequences were denatured at 99° C. for 5 min and then 45° C. for 5 min and hybridized to oligonucleotide arrays comprised of a large number of human genes (HG-U95A or HG-U133A, Affymetrix, Santa Clara, Calif., USA). Arrays were hybridized for 16 h at 45° C. with rotation at 60 rpm. After hybridization, the hybridization mixtures were removed and stored, and the arrays were washed and stained with Streptavidin R-phycoerythrin (Molecular Probes) using GeneChip Fluidics Station 400 and scanned with a Hewlett Packard GeneArray Scanner following the manufacturer's instructions. These hybridization and wash conditions are collectively referred to as “nucleic acid array hybridization conditions.”

### Example 3

### Determination of Gene Expression Frequencies and Processing of Expression Data

Array images were processed using the Affymetrix MicroArray Suite software (MAS) such that raw array image data (.dat) files produced by the array scanner were reduced to probe feature-level intensity summaries (.cel files) using the desktop version of MAS. Using the Gene Expression Data System (GEDS) as a graphical user interface, users provide a sample description to the Expression Profiling Information and Knowledge System (EPIKS) Oracle database and associate the correct cel file with the description. The database processes then invoke the MAS software to create probeset summary values; probe intensities are summarized for each message using the Affymetrix Average Difference algorithm and the Affymetrix Absolute Detection metric (Absent, Present, or Marginal) for each probeset. MAS is also used for the first pass normalization by scaling the trimmed mean to a value of 100. The database processes also calculate a series of chip quality control metrics and store all the raw data and quality control calculations in the database.

Data analysis and absent/present call determination was performed on raw fluorescent intensity values using MAS software (Affymetrix). “Present” calls are calculated by MAS software by estimating whether a transcript is detected in a sample based on the strength of the gene's signal compared to background. The “average difference” values for each transcript were normalized to “frequency” values using the scaled frequency normalization method (Hill, et al., GENOME BIOL, 2:research0055.1-0055.13 (2001)) in which the average differences for 11 control cRNAs with known abundance spiked into each hybridization solution were used to generate a global calibration curve. This calibration was then used to convert average difference values for all transcripts to frequency estimates, stated in units of parts per million ranging from 1:300,000 (˜3 parts per million (ppm)) to 1:1000 (1000 ppm). The normalization refers the average difference values on each chip to a calibration curve constructed from the average difference values for the 11 control transcripts with known abundance that were spiked into each hybridization solution. In many instances, the normalization method utilizes a trimmed-mean normalization, followed by fitting of a pooled standard curve across all chips, which is used to compute “frequency” values and per-chip sensitivity estimates. The resulting metric is referred to as a scaled frequency and normalizes between all arrays.

Genes that did not have any relevant information were excluded from the data comparison. In comparisons of disease-free PBMCs with RCC PBMCs, this was accomplished using two data reduction filters: 1) any gene that was called Absent on all GeneChips (as determined by the Affymetrix Absolute Detection metric in MAS) was removed from the dataset; 2) any gene that was expressed at a normalized frequency of <10 ppm on all GeneChips was removed from the dataset to ensure that any gene kept in the analysis set was detected at a frequency of at least 10 ppm at least once. The total number of probe sets in the analysis after these filtering steps were performed was 5,469. For some multivariate prediction analyses more stringent data reduction filters were used (25% P, and average frequency>5 ppm) in order to decrease the likelihood that low level or infrequently detected transcripts would be identified.

### Example 4

### Pearson's-Based Assessment of Outlier Samples

To identify outlier samples, the square of the pairwise Pearson correlation coefficient (r2) among all pairs of samples was computed using Splus (Version 5.1). Specifically, the computation was started from the G×S matrix of expression values, where G is the total number of probesets and S is the total number of samples. r2-values between samples in this matrix were calculated. The result was a symmetric S×S matrix of r2-values. This matrix measures the similarity between each sample and all other samples in the analysis. Since all of these samples come from human PBMCs harvested according to common protocols, the expectation is that the correlation coefficients reveal a high degree of similarity in general (i.e., the expression levels of the majority of the transcript sequences are similar in all samples analyzed). To summarize the similarity of samples, the average of the r2-values between all MAS signals of each sample and the other samples in the study was calculated and plotted in a heat map to facilitate rapid visualization. The closer the value of average r2 is to 1, the more alike the sample is to the other samples within the analysis. Low average r2-values indicate that the gene expression profile of the sample is an “outlier” in terms of overall gene expression patterns. Outlier status can indicate either that the sample has a gene expression profile that deviates significantly from the other samples within the analysis, or that the technical quality of the sample was of inferior quality.

### Example 5

### Clinical Study Protocol Summary

PBMCs were isolated from peripheral blood of 20 disease-free volunteers (12 females and 8 males) and 45 renal cell carcinoma patients (18 females and 27 males) participating in the phase II study. Consent for the pharmacogenomic portion of the clinical study was received and the project was approved by the local Institutional Review Boards at the participating clinical sites. The RCC tumors were classified at each site as conventional (clear cell) carcinomas (24), granular (1), papillary (3), or mixed subtypes (7). Classifications for ten tumors were not identified. The 45 patients who signed informed consent for pharmacogenomic analysis of baseline PBMC expression profiles were also scored by the multivariate assessment method of Motzer. Of the consented patients enrolled in this study, 6 were assigned a favorable risk assessment, 17 patients possessed an intermediate risk score, and 22 patients received a poor prognosis classification in this study.

Patients with advanced cases of RCC were treated with one of 3 doses of CCI-779 (25 mg, 75 mg, 250 mg) administered as a 30 minute IV infusion once weekly for the duration of the trial. Clinical staging and size of residual, recurrent or metastatic disease were recorded prior to treatment and every 8 weeks following initiation of CCI-779 therapy. Tumor size was measured in centimeters and reported as the product of the longest diameter and its perpendicular. Measurable disease was defined as any bidimensionally measurable lesion where both diameters>1.0 cm by CT-scan, X-ray or palpation. Tumor responses (complete response, partial response, minor response, stable disease or progressive disease) were determined by the sum of the products of the perpendicular diameters of all measurable lesions. The two main clinical outcome measures utilized in the present pharmacogenomic study were time to progression (TTP) and survival or time to death (TTD). TTP was defined as the interval from the date of initial CCI-779 treatment until the first day of measurement of progressive disease, or censored at the last date known as progression-free. Survival or TTD was defined as the interval from date of initial CCI-779 treatment to the time of death, or censored at the last date known alive.

### Example 6

### Statistical Analyses

Unsupervised hierarchical clustering of genes and/or arrays on the basis of similarity of their expression profiles was performed using the procedure of Eisen, et al., PROC NATL ACAD SCI U.S.A., 95:14863-14868 (1998). In these analyses only those transcripts meeting a non-stringent data reduction filter were used (at least 1 present call, at least 1 frequency across the data set of greater than or equal to 10 ppm). Expression data were log transformed and standardized to have a mean value of zero and a variance of one, and hierarchical clustering results were generated using average linkage clustering with an uncentered correlation similarity metric.

To identify transcripts changing over time in all CCI-779 treated patients with complete time courses (n=21), a standard ANOVA was used and average fold changes between various time points (baseline, 8 weeks, 16 weeks) were calculated.

To identify transcripts exhibiting changes correlated with clinical outcome, correlations between the continuous measures of clinical outcome (TTP and TTD) and changes in gene expression from baseline to 8 or 16 weeks were computed for each transcript using the Spearman's rank correlation. Alterations in gene expression data between baseline and 8 or 16 weeks were also assessed with censored measures of clinical outcomes (TTP, TTD) using a Cox proportional hazards regression model.

Survival data of various groups of patients were assessed by Kaplan Meier analysis, and significance was established using a Wilcoxon test.

The foregoing description of the present invention provides illustration and description, but is not intended to be exhaustive or to limit the invention to the precise one disclosed. Modifications and variations are possible consistent with the above teachings or may be acquired from practice of the invention. Thus, it is noted that the scope of the invention is defined by the claims and their equivalents.

What is claimed is:

1. A method for prognosis, or evaluation of the effectiveness of a treatment, of a solid tumor in a patient of interest, said method comprising:
1) detecting a change in expression level of at least one gene in peripheral blood cells of the patient of interest during the course of the treatment of the patient, wherein said changes in patients who have the same solid tumor and receive the same treatment as the patient of interest are correlated with clinical outcomes of said patients under a correlation model; and
2) comparing said change in the patient of interest to a reference change, wherein the difference between said change in the patient of interest and the reference change is indicative of the prognosis, or the effectiveness of the treatment, of said solid tumor in the patient of interest.

2. The method of claim 1, wherein said correlation model is a Cox proportional hazards model.

3. The method of claim 2, wherein said solid tumor is renal cell carcinoma (RCC), and the treatment comprises a CCI-779 therapy.

4. The method of claim 3, wherein said change in the patient of interest is a change between an expression level of said at least one gene in peripheral blood cells of the patient of interest at a specified time after initiation of the treatment of the patient and a baseline expression level of said at least one gene in peripheral blood cells of the patient of interest, and wherein said reference change is a change between an expression level of said at least one gene in peripheral blood cells of a reference patient at said specified time after initiation of the treatment of the reference patient and a baseline expression level of said at least one gene in peripheral blood cells of the reference patient, said reference patient having said solid tumor.

5. The method of claim 4, wherein said specified time is about 16 weeks after initiation of the treatment.

6. The method of claim 4, wherein said peripheral blood cells comprise whole blood cells.

7. The method of claim 4, wherein said peripheral blood cells comprise enriched peripheral blood mononuclear cells (PBMCs).

8. The method of claim 4, wherein said at least one gene has a hazard ratio of less than 1, and a greater value of said change in the patient of interest as compared to said reference change is suggestive that the patient of interest has a better prognosis than the reference patient, and a lesser value of said change in the patient of interest as compared to said reference change is suggestive that the patient of interest has a poorer prognosis than the reference patient.

9. The method of claim 4, wherein said at least one gene has a hazard ratio of greater than 1, and a greater value of said change in the patient of interest as compared to said reference change is suggestive that the patient of interest has a poorer prognosis than the reference patient, and a lesser value of said change in the patient of interest as compared to said reference change is suggestive that the patient of interest has a better prognosis than the reference patient.

10. The method of claim 4, wherein each of said at least one gene is selected from the genes listed in Tables 4A, 4B, 5A or 5B.

11. The method of claim 2, wherein said reference change has an empirically or experimentally determined value.

12. The method of claim 11, wherein said solid tumor is RCC, and the treatment comprises a CCI-779 therapy, and wherein said change in the patient of interest is a change between an expression level of said at least one gene in peripheral blood cells of the patient of interest at a specified time after initiation of the treatment of the patient and a baseline expression level of said at least one gene in peripheral blood cells of the patient.

13. The method of claim 12, wherein said specified time is about 16 weeks after initiation of the treatment.

14. The method of claim 12, wherein each of said at least one gene is selected from the genes listed in Tables 4A, 4B, 5A or 5B, and said peripheral blood cells comprise whole blood cells or enriched PBMCs.

15. The method of claim 12, wherein said at least one gene has a hazard ratio of less than 1, and a greater value of said change in the patient of interest as compared to said reference change is suggestive of a good prognosis of the patient of interest, and a lesser value of said change in the patient of interest as compared to said reference change is suggestive of a poor prognosis of the patient of interest.

16. The method of claim 12, wherein said at least one gene has a hazard ratio of greater than 1, and a greater value of said change in the patient of interest as compared to said reference change is suggestive of a poor prognosis of the patient of interest, and a lesser value of said change in the patient of interest as compared to said reference change is suggestive of a good prognosis of the patient of interest.

17. The method of claim 12, wherein said reference change is an average change between expression levels of said at least one gene in peripheral blood cells of reference patients at said specified time after initiation of the treatment of said reference patients and the corresponding baseline expression levels of said at least one gene in peripheral blood cells of said reference patients, each said reference patient having said solid tumor.

18. A method for prognosis, or evaluation of the effectiveness of a treatment, of a solid tumor in a patient of interest, said method comprising:
1) detecting a change in expression profile of two or more genes in peripheral blood cells of the patient of interest during the course of the treatment of the patient, wherein said changes in patients who have the same solid tumor and receive the same treatment as the patient of interest are correlated with clinical outcomes of said patients under a correlation model; and
2) comparing said change in the patient of interest to a reference change, wherein the difference between said change in the patient of interest and the reference change is indicative of the prognosis, or the effectiveness of the treatment, of said solid tumor in the patient of interest.

19. A kit for prognosis or evaluation of the effectiveness of a treatment of a solid tumor in a patient of interest, said kit comprising one or more probes for an expression product of a gene selected from the genes listed in Tables 4A, 4B, 5A or 5B.

20. A method for identifying markers that are prognostic of a solid tumor, comprising:
1) detecting changes in gene expression profiles in peripheral blood cells of patients during the course of an anti-cancer treatment of said patients, each said patient having said solid tumor; and
2) identifying genes whose said changes in said patients are correlated with clinical outcomes of said patients under a correlation model.
